(19) United States
(12) Patent Application Publication
(10) Pub. No.: US 2025/0090552 A1
(43) Pub. Date: Mar. 20, 2025

(54) MESALAMINE PHARMACEUTICAL FORMULATIONS AND METHODS OF USE THEREOF

(71) Applicant: Intact Therapeutics, Inc., Belmont, CA (US)

(72) Inventor: Ravinder D. Pamnani, Los Altos, CA (US)

(21) Appl. No.: 18/832,003

(22) PCT Filed: Feb. 6, 2023
(86) PCT No.: PCT/US2023/012410

§ 371 Date: Jul. 22, 2024

Related U.S. Application Data
(60) Provisional application No. 63/306,942, filed on Feb. 4, 2022.

(51) Int. Cl. A61K 31/606; A61K 9/06; A61K 9/107; A61K 47/10; A61K 47/24

(52) U.S. Cl. CPC A61K 31/606; A61K 9/06; A61K 9/107; A61K 47/10; A61K 47/24

(57) ABSTRACT

The invention provides methods and compositions for local administration of therapeutic agents to the rectum or colon, such as by enema. The methods and compositions are useful for treatment of inflammatory bowel disease, including Crohn's disease and ulcerative colitis.

### RELATED APPLICATIONS

The present application claims the benefit of and priority to U.S. provisional patent application Ser. No. 63/306,942, filed Feb. 4, 2022, the content of which is incorporated by reference herein in its entirety.

### FIELD OF THE INVENTION

The invention relates generally to a pharmaceutical formulation of mesalamine, a kit for providing the invention to a subject, and methods of its use by a subject.

### BACKGROUND

Inflammatory bowel disease (IBD) afflicts about 3 million Americans and over 11 million people worldwide. IBD is a group of inflammatory conditions of the gastrointestinal tract, including the mouth, esophagus, stomach, small intestine, and colon, with Crohn's disease and ulcerative colitis being the predominant forms. IBD causes debilitating symptoms, such as abdominal pain, diarrhea, rectal bleeding, cramping, weight loss, and anemia, and can be fatal if left untreated.

Anti-inflammatory agents, such as mesalamine (also called mesalazine or 5-aminosalicylic acid (5-ASA)), or corticosteroids, such as budesonide or hydrocortisone, are often used to treat IBD. However, despite the existence of suitable pharmacological agents to combat many forms of IBD, treatment of IBD is hampered by the difficulties of administering such agents. For example, when drugs such as mesalamine or budesonide are administered orally, most of the active agent is metabolized as it passes through the gastrointestinal tract or eliminated through bulk transit. Consequently, oral formulations generally fail to achieve therapeutic concentrations of the drug in the distal colon, the site of lesions in ulcerative colitis and many cases of Crohn's disease. Formulations designed to overcome this obstacle by preventing release of the drug in the stomach are hindered by other problems, such as incomplete release, release in the proximal rather than distal colon, release of toxic metabolites, and high patient-to-patient variability. Another concern is that oral or parenteral administration of immunosuppressive agents increases the risk of malignancies and infections.

Current methods of local administration are plagued by their own set of problems. The GI tract, including the colon, is designed to continuously move consumed content through the body while absorbing nutrients. As a result, therapeutic agents administered locally, i.e., directly to the surface of the colon, tend to get rapidly cleared from target tissue during transit of digested material through the bowel. Because treatment of inflamed or infected GI tissue requires sustained exposure of such tissue to therapeutic agents, frequent administration (e.g., once daily or multiple times per day) is often necessary to achieve the full therapeutic benefit of a locally administered agent. As a result, many patients fail to comply with a prescribed dosing regimen.

Other barriers to effective local treatment of IBD stem from the mode of administration. Enemas permit delivery of therapeutic agents to the entirety of the descending colon. However, enema-based treatments typically require patients to retain a substantial volume of liquid (e.g., 60-100 ml) in the colon for an extended period in multiple daily administrations, an unpleasant exercise that further hampers patient compliance. Suppositories and foams are less inconvenient than enemas but generally fail to deliver agents beyond the rectum and sigmoid colon, respectively. One of the primary symptoms of IBD is urgency and diarrhea, and a bowel movement will clear out the enema, foam, or suppository formulations, thereby limiting efficacy. Consequently, existing methods for delivering therapeutic agents are inadequate to treat many forms of IBD, and millions of people continue to suffer from conditions such as Crohn's disease and ulcerative colitis.

### SUMMARY

The present invention includes improved thermogelling compositions and formulations and methods for using them to treat inflammatory conditions of the gastrointestinal tract. The compositions of the invention include an active ingredient, at least one grade of thermogelling polymer (wherein, if present, each grade is present in a different concentration), a lipid, and a solubilizer of the lipid. The invention also provides a kit that includes a first container comprising a first composition comprising the active ingredient; and a second container comprising a second composition comprising at least one grade of thermogelling polymer, a lipid, and a solubilizer of the lipid. The invention further provides a method of treating a condition in a subject, the method comprising the steps of receiving a kit comprising a first container comprising a first composition comprising the active ingredient; and a second container comprising a second composition comprising at least one grade of thermogelling polymer, a lipid, and a solubilizer of the lipid, mixing the first and second compositions to form a final formulation, and administering the final formulation.

In certain embodiments, compositions are in a liquid state near room temperature (e.g., 20-25° C.) and transition to a gel state near body temperature (e.g., 32-37° C.). This enables the invention to be stored as a liquid and provide the penetration/access advantages of a liquid enema. However, when introduced to body temperature, the composition gels, making it easier for a subject to retain the enema thereby providing the retention advantages of foams or suppository preparations. Retention of the enema is critical to the therapeutic effect of the enema. However, the invention also provides increased absorption of the active ingredient because of the other agents included in the formulation.

The invention provides utility in favor of currently used compositions because the stability of the active ingredient can be improved by the addition of some of the other agents. The kit's separation of the active ingredient from the other agents until the subject is ready to administer the agent solves a stability problem associated with prior formulations and makes the preparation more commercially viable because of a longer shelf life.

The active ingredient in an example embodiment is mesalamine. Mesalamine breaks down in an aqueous environment, reducing the stability and shelf life of mesalamine, particularly within formulations of the invention. Furthermore, mesalamine can cause polymers such as poloxamer 407 and 188, also present in an embodiment of the invention, themselves break down in the formulation. Therefore, keeping the active ingredient, such as mesalamine, in a separate container and out of aqueous solution (e.g., dry) until extemporaneously compounded by the end user, patient or caregiver, allows for dramatically improved shelf stability and supply chain durability compared to a formulation that is provided with mesalamine and poloxamer already present together. It also provides greater efficacy with reduced side effects because the formulation is more likely to provide the desired dose to the patient, and the formulation does not need to incorporate additional stabilizers or excipients that may cause adverse events.

Another issue solved by the present invention is the ability to transport and ship the thermogelling product, which can be subjected to increased temperatures if not kept in strict temperature control. By keeping the active ingredient separate, if the gelling should occur in transportation, the quality of the active ingredient will not be affected as it would be if already mixed with the other agents.

The presence of the lipid and solubilizer of the lipid help to regulate the temperature at which the polymer transitions from liquid to gel. Too low of a gelling temperature and the composition can form a gel on the shelf, compromising the product. Too high, and body temperature may not be enough to cause the product to gel, eliminating the advantages this composition has over liquid enemas.

Embodiments of the invention may use mesalamine as the active ingredient, poloxamer 407, poloxamer 188 as the grades of thermogelling polymers present in different concentrations, phosphatidylcholine as the lipid, and diethylene glycol monoethyl ether as the solubilizing agent.

In an embodiment of the invention, the concentration of mesalamine is between about 0.5-15% w/v of the composition; in another embodiment, the concentration of mesalamine is between about 6-8% w/v of the composition. In an embodiment of the invention, the concentration of poloxamer 407 is between about 10-16% w/v of the composition; in another embodiment, the concentration of poloxamer 407 is between about 12-13.5% w/v of the composition. In an embodiment of the invention, the concentration of poloxamer 188 is between about 0.001-1% w/v of the composition. In an embodiment of the invention, the concentration of phosphatidylcholine is between about 0.0014% w/v of the composition; in another embodiment of the invention, the concentration of phosphatidylcholine is 1.5-2.5% w/v of the composition. In an embodiment of the invention, the concentration of diethylene glycol monoethyl ether is between 5-15% w/v of the composition; in another embodiment of the invention, the concentration of diethylene glycol monoethyl ether is between about 8-10% w/v of the composition.

The invention further discloses an embodiment as a kit, the kit comprising a first container comprising a first composition comprising the active ingredient; and a second container comprising a second composition comprising at least one grade of thermogelling polymer wherein, if present, the grades are present in different concentrations, a lipid, and a solubilizer of the lipid.

A further embodiment of the invention is a method of treating a condition in a subject, the method comprising the steps of receiving a kit comprising a first container comprising a first composition comprising the active ingredient; and a second container comprising a second composition comprising at least one grade of thermogelling polymer wherein, if present, the grades are present in different concentrations, a lipid, and a solubilizer of the lipid, mixing the first and second compositions to form a final formulation, and administering the final formulation. The condition may be an irritable bowel disorder, or ulcerative colitis. In an embodiment of the invention, the subject administers the formulation topically, in one embodiment as an enema.

According to a method of the invention, the subject performs the steps of mixing and administering. Mixing may comprise adding the second composition to the first composition and shaking for at least 30 seconds to suspend the formulation. Mixing may comprise adding the second composition to the first composition and shaking for at least 15 seconds to suspend the formulation. Mixing may comprise adding the second composition to the first composition and shaking for at least 10 seconds, holding at rest for 1 minute, then shaking again for 10 more seconds to suspend the formulation. Mixing may comprise adding the first composition to the second composition and shaking for at least 10 seconds, holding at rest for 1 minute, then shaking again for 10 more seconds to suspend the formulation.

### BRIEF DESCRIPTION OF THE DRAWINGS

FIG. 1 diagrams the layout of a study described in the Examples.

FIG. 2 shows study period dosing and a PK sampling timeline for the study of FIG. 1.

FIG. 3 shows plasma PK for the test compound (INT-001) vs. the reference compound (ROWASA) of the study of FIG. 1. FIG. 3A shows mesalamine while FIG. 3B shows n-acetyl-mesalamine.

FIG. 4 shows mesalamine reference data for the study of FIG. 1.

FIG. 5 shows data for the test compound of the study of FIG. 1.

FIG. 6 shows mean 5-ASA in stool for the test and reference compounds in the study of FIG. 1.

FIG. 7 shows mean n-ac-5-ASA in stool for the test and reference compounds in the study of FIG. 1.

FIG. 8 shows time to first stool after administration of the reference and the test compounds for six example test subjects in the study of FIG. 1.

FIG. 9 shows stability data for various exemplary compound formulations.

FIG. 10 shows poloxamer stability for lipoid S100.

FIG. 11 shows poloxamer stability for p90G.

FIG. 12 shows gelation temperatures for various compounds in an excipient compatibility study.

FIG. 13 shows gelation temperatures for various lipid combinations.

FIG. 14 shows gelation temperatures for Poloxamer 188.

FIG. 15 shows gelation temperatures for lipoid S 100 compositions.

FIG. 16 shows gelation temperatures for phospholipon 90 G compositions FIG. 17 shows rheology results for composition 278.

FIG. 18 shows rheology results for composition 279.

FIG. 19 shows rheology results for composition 280.

FIG. 20 shows rheology results for composition 278 vs. composition 291.

FIG. 21 shows rheology results for composition 279 vs. composition 292.

FIG. 22 shows rheology results for composition 280 vs. composition 293.

### DETAILED DESCRIPTION

The invention comprises a composition comprising an active ingredient, at least one grade of thermogelling polymer, a lipid, and a solubilizer of the lipid. It further comprises a kit comprising a first container comprising a first composition comprising the active ingredient; and a second container comprising a second composition comprising at least one grade of thermogelling polymer, a lipid, and a solubilizer of the lipid. The invention further comprises a method of treating a condition in a subject, the method comprising the steps of receiving a kit comprising a first container comprising a first composition comprising the active ingredient; and a second container comprising a second composition comprising at least one grade of thermogelling polymer, a lipid, and a solubilizer of the lipid, mixing the first and second compositions to form a final formulation, and administering the final formulation.

The active ingredient may be an aminosalicylate. The aminosalicylate may be 5-ASA, 4-ASA, azodisalicylate, balsalazide, ipsalazide, olsalazine, or sulfasalazine.

An embodiment of the invention is comprised by mesalamine as the active ingredient. Mesalamine is known variously as mesalazine, 5-aminosalicylic acid, 5-ASA. Mesalamine has a structure of:

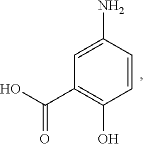 [SMILES: Nc1ccc(O)c(C(=O)O)c1]

and a chemical formula of C₇H₇NO₃. Mesalamine is the active moiety of sulfasalazine, which is metabolized to sulfapyridine and mesalamine. Mesalamine is known as a disease modifying anti-rheumatic drug (DMARD), but its precise mechanism of action is unknown. Mesalamine may reduce activity of cyclooxygenase and lipoxygenase, and thus reduce prostaglandin presence, which in turn has an anti-inflammatory effect. Current mesalamine treatments administer the drug locally within the gastrointestinal lumen, either through enteric coated or other delayed release oral dosage forms, or rectally as an enema. In an embodiment of the invention, the concentration of mesalamine is between about 0.5-15% w/v of the composition; in another embodiment, the concentration of mesalamine is between about 6-8% w/v of the composition.

The invention may comprise either one or a plurality of grades of thermogelling polymer, wherein each grade is present at a different concentration.

A preferred embodiment of the invention may comprise poloxamer 407 as the or one of the grades of thermogelling polymer. Poloxamer 407 is a copolymer used as a hydrophilic non-ionic surfactant. As a surfactant, it lowers surface tension and enables suspension and emulsification of, for example, lipophilic solids in hydrophilic liquids, or vice versa. It has a chemical formula of C₅₇₂H₁₁₄₆O₂₅₉ and has two hydrophilic blocks of polyethylene glycol with about 101 repeats surrounding a block of polypropylene glycol of 56 repeats. In an embodiment of the invention, the concentration of poloxamer 407 is between about 10-16% w/v of the composition; in another embodiment, the concentration of poloxamer 407 is between about 12-13.5% w/v of the composition.

Another embodiment of the invention may comprise poloxamer 188 as the or one of the grades of thermogelling polymer. Poloxamer 188 is also a copolymer and is another surfactant molecule. It has a chemical formula of C₈H₁₈O₃ and is a copolymer of ethylene oxide and propylene oxide. In an embodiment of the invention, the concentration of poloxamer 188 is between about 0.001-1% w/v of the composition.

An embodiment of the invention may comprise a lipid. Lipids are a major group of biomolecules that are typically hydrocarbons. Lipids are normally not dissolvable in water. Phospholipids are a class of lipid where there are hydrophobic tails protruding from a hydrophilic head of the molecule. Phospholipids are naturally occurring and present in cell membranes.

An embodiment of the invention may comprise phosphatidylcholine as the lipid. Phosphatidylcholine is a type of phospholipid that has choline as a head group; it is a common component of biological membranes and acts as a surfactant. Phosphatidylcholine may be produced commercially by purifying naturally occurring phosphatidylcholine. Commercially available phosphatidylcholine is, in an embodiment of the invention, LIPOID S 100 (phosphatidylcholine from soybean with agglomerates, Lipoid GmbH). In an embodiment of the invention, the concentration of phosphatidylcholine is between about 0.0014% w/v of the composition; in another embodiment of the invention, the concentration of phosphatidylcholine is 1.5-2.5% w/v of the composition.

An embodiment of the invention may comprise a solubilizer of the lipid. A solubilizing agent acts as a surfactant and increases the solubility of one agent in another. For example, lipophilic substances that may not solubilize in an aqueous solution may be solubilized by solubilizing agents that act as a surfactant and decrease surface tension between the solute and solvent.

An embodiment of the invention may comprise diethylene glycol monoethyl ether as the solubilizing agent. Diethylene glycol monoethyl ether has a chemical formula of CH₃CH₂OCH₂CH₂OCH₂CH₂OH, has an IUPAC name of 2-(2-Ethoxyethoxy)ethan-1-ol and demonstrates activity as a solvent. Diethylene glycol monoethyl ether is sold under many trade names, including Transcutol (Millipore Sigma KGaA). In an embodiment of the invention, the concentration of diethylene glycol monoethyl ether is between 5-15% w/v of the composition; in another embodiment of the invention, the concentration of diethylene glycol monoethyl ether is between about 8-10% w/v of the composition.

In another embodiment of the invention, the invention is provided as a kit, the kit comprising a first container comprising a first composition comprising the active ingredient; and a second container comprising a second composition comprising a plurality of grades of thermogelling polymer wherein the grades are present in different concentrations, a lipid, and a solubilizer of the lipid.

A further embodiment of the invention is a method of treating a condition in a subject, the method comprising the steps of receiving a kit comprising a first container comprising a first composition comprising the active ingredient; and a second container comprising a second composition comprising a plurality of grades of thermogelling polymer wherein the grades are present in different concentrations, a lipid, and a solubilizer of the lipid, mixing the first and second compositions to form a final formulation, and administering the final formulation. The method may include providing the kit locally to the colon of a subject. The methods are useful for the treating of gastrointestinal disorders, such as irritable bowel disorder, ulcerative colitis, or Crohn's disease.

In an embodiment of the invention, the composition is a liquid at 20-25° C. and transitions to a gel at 32-37° C. This allows the provision of the invention to a subject in liquid stable form, and then when administered to a subject rectally by enema, the temperature of the mixture would increase to the body temperature of the subject. For the invention, this causes gelation when the composition is introduced into the colon, making the retention of the enema easier for the subject. Retention of the enema is critical to the absorption of the mesalamine and therefore the efficacy thereof.

According to one method of the invention, the subject performs the steps of mixing and administering. Mixing may comprise adding the second composition to the first composition and shaking for at least 30 seconds to suspend the formulation. Mixing may comprise adding the second composition to the first composition and shaking for at least 15 seconds to suspend the formulation. Mixing may comprise adding the second composition to the first composition and shaking for at least 10 seconds, holding at rest for 1 minute, then shaking again for 10 more seconds to suspend the formulation. Mixing may comprise adding the first composition to the second composition and shaking for at least 10 seconds, holding at rest for 1 minute, then shaking again for 10 more seconds to suspend the formulation.

### Dosing

A person having skill in the art will determine the dosing interval for the method. A dosing regimen may include one or more of a dosage, frequency of administration, mode of administration, and duration. A multiple-dose regimen may differ in dosage, frequency of administration, mode of administration, or any combination thereof.

The frequency of administration may be defined by the interval between doses. For example and without limitation, the interval between doses may be about 6 hours, about 8 hours, about 12 hours, about 15 hours, about 24 hours, about 36 hours, about 48 hours, about 60 hours, about 72 hours, about 84 hours, about 96 hours, about 5 days, about 6 days, about 7 days, about 8 days, about 10 days, about 12 days, about 14 days, about 3 weeks, about 4 weeks, at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 10 days, at least 12 days, at least 14 days, at least 3 weeks, at least 4 weeks, greater than 6 hours, greater than 8 hours, greater than 12 hours, greater than 15 hours, greater than 24 hours, greater than 36 hours, greater than 48 hours, greater than 60 hours, greater than 72 hours, greater than 84 hours, greater than 96 hours, greater than 5 days, greater than 6 days, greater than 7 days, greater than 8 days, greater than 10 days, greater than 12 days, greater than 14 days, greater than 3 weeks, or greater than 4 weeks.

A dosing regimen may include one or more modes of administration. The mode of administration may be suitable for local administration or for systemic administration. For example and without limitation, modes of local administration include topical administration and rectal administration, e.g., via enemas, suppositories, foams, and other methods of delivery via the anus. For example and without limitation, modes of systemic administration include oral, enteral, parenteral, by injection, and by infusion.

In methods involving multiple phases, e.g., two or more phases of treatment, the phases may differ by any relevant parameter. For example, and without limitation, the phases may differ in one or more of duration, dosage, frequency of dose administration, mode of administration, route of administration, or therapeutic composition. The first phase of treatment may have a longer or shorter duration than the second phase of treatment. The first phase of treatment may have a higher or lower dosage of an agent than the second phase of treatment. The first phase of treatment may have a smaller or larger interval between dose administrations than the second phase of treatment. The first and second phases of treatment have the same mode of administration, or they may have different modes of administration. The first and second phases of treatment have the same route of administration, or they may have different routes of administration. The first and second phases of treatment employ the same composition, or they may employ different compositions. One phase of treatment may employ a single therapeutic agent, and another phase may employ a combination of therapeutic agents. The first and second phases of treatment may employ different combinations of therapeutic agents.

The subject may be an animal, such as a mammal. A mammalian subject may be, nonexclusively, a human, mouse, or rat.

The different dosing regimens may achieve distinct therapeutic goals. For example, the first dosing regimen may induce remission of the condition, and the second dosing may maintain remission of the condition.

Induction therapy is typically used to treat an acute phase of the condition or provide relief from symptoms associated with an acute phase. An acute phase of a condition may have one or more of the following features: abrupt onset, short duration, rapid progression, the need for urgent care, elevated levels of diagnostic markers. Acute phases of certain conditions, such as IBD, are known as “flare-ups”.

Maintenance therapy, on the other hand, generally prevents the condition or its symptoms from recurring. Maintenance therapy may be used for treatment of any non-acute phase of a condition, i.e., any phase of a condition that does not meet one or more criteria of an acute phase. Thus, maintenance therapy is usually long-term and continues even when the patient does not experience symptoms.

Methods of the invention may include providing an agent locally as described herein without the use of another form of therapy. Alternatively, methods of the invention may include providing an agent locally as described herein in combination with another form of therapy. For example and without limitation, the second form of therapy may differ in the agent, dosing regimen, dosage, dosing interval, mode of administration, route of administration, or any combination of the aforementioned elements. For example, the second form of therapy may include administration of an agent non-locally, e.g., systemically or orally. The second form of therapy may be performed prior to, concurrently with, or after, providing an agent locally as described herein. Each therapeutic method may independently induce remission of the condition, maintain remission of the condition, or both.

### Treating GI Conditions Following an Acute Phase

Embodiments of the invention include methods of treating a GI condition by providing an agent locally to the rectum or colon of a subject following an acute phase or flare of the condition. The treatment may maintain the GI condition in a reduced state. For example, the treatment may maintain remission of the condition. Additionally, or alternatively, the methods may include treating a GI condition by providing an agent locally to the upper GI tract, e.g., mouth, esophagus, or stomach, of a subject following an acute phase or flare of the condition.

As indicated above, an acute phase or flare of a condition may have one or more of the following features: abrupt onset, short duration, rapid progression, the need for urgent care, elevated levels of diagnostic markers. Thus, treatments following an acute phase may entail treatment of any post-acute phase of a condition that does not meet one or more criteria of an acute phase.

The methods may include administering an agent locally according to a dosing regimen. The dosing regimen may include any of the elements described above in relation to dosing regimens, such as a dosage, frequency of administration, mode of administration, and duration.

As indicated above, the frequency of administration may be defined by the interval between doses. For example and without limitation, the interval between doses may be about 6 hours, about 8 hours, about 12 hours, about 15 hours, about 24 hours, about 36 hours, about 48 hours, about 60 hours, about 72 hours, about 84 hours, about 96 hours, about 5 days, about 6 days, about 7 days, about 8 days, about 10 days, about 12 days, about 14 days, about 3 weeks, about 4 weeks, at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 10 days, at least 12 days, at least 14 days, at least 3 weeks, at least 4 weeks, greater than 6 hours, greater than 8 hours, greater than 12 hours, greater than 15 hours, greater than 24 hours, greater than 36 hours, greater than 48 hours, greater than 60 hours, greater than 72 hours, greater than 84 hours, greater than 96 hours, greater than 5 days, greater than 6 days, greater than 7 days, greater than 8 days, greater than 10 days, greater than 12 days, greater than 14 days, greater than 3 weeks, or greater than 4 weeks.

As described above, a dosing regimen may include one or more modes of administration. The mode of administration may be suitable for local administration or for systemic administration. For example, and without limitation, modes of local administration include topical administration and rectal administration, e.g., via enemas, suppositories, foams, and other methods of delivery via the anus. For example, and without limitation, modes of systemic administration include oral, enteral, parenteral, by injection, and by infusion.

The subject may be any type of subject, as described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

### Treating GI Conditions Using Extended Intervals Between Doses

Embodiments of the invention include treating a GI condition by repeatedly providing an agent locally to the rectum or colon of a subject in which the doses are separated by extended intervals. Additionally, or alternatively, the methods may include treating a GI condition by repeatedly providing an agent locally to the upper GI tract, e.g., mouth, esophagus, or stomach, of a subject in which the doses are separated by extended intervals.

A problem with prior methods of topical administration of therapeutic agents to the colon is that the poor retention of the agent in the colon following bowel movements necessitates frequent re-administration of the agent via enema, the inconvenience of which leads to low rates of patient compliance with prescribed dosing regimens. See, e.g., Boyle, et al, Adherence to Rectal Mesalamine in Patients with Ulcerative Colitis, Inflamm. Bowel Dis. 2015 December; 21(12):2873-8. doi: 10.1097/MIB.0000000000000562, the contents of which are incorporated herein by reference. Due to the superior retention of therapeutic agents in the colon using the compositions and methods of the invention, embodiments of the invention allow subjects extended intervals between administrations of enemas, leading to better patient compliance.

The interval between doses may be a defined period. For example and without limitation, the interval between doses may be about 6 hours, about 8 hours, about 12 hours, about 24 hours, about 36 hours, about 48 hours, about 60 hours, about 72 hours, about 84 hours, about 96 hours, about 5 days, about 6 days, about 7 days, at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, greater than 6 hours, greater than 8 hours, greater than 12 hours, greater than 24 hours, greater than 36 hours, greater than 48 hours, greater than 60 hours, greater than 72 hours, greater than 84 hours, greater than 96 hours, greater than 5 days, greater than 6 days, or greater than 7 days.

The agent may be retained in the colon for a defined period. For example and without limitation, the agent may be retained in the colon for at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, greater than 6 hours, greater than 8 hours, greater than 12 hours, greater than 24 hours, greater than 36 hours, greater than 48 hours, greater than 60 hours, greater than 72 hours, greater than 84 hours, greater than 96 hours, greater than 5 days, greater than 6 days, or greater than 7 days.

The agent may exert or maintain a therapeutic effect in the colon for a defined period. For example and without limitation, the agent may exert or maintain a therapeutic effect in the colon for at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, greater than 6 hours, greater than 8 hours, greater than 12 hours, greater than 24 hours, greater than 36 hours, greater than 48 hours, greater than 60 hours, greater than 72 hours, greater than 84 hours, greater than 96 hours, greater than 5 days, greater than 6 days, or greater than 7 days.

The methods may include administering an agent locally according to a dosing regimen. The dosing regimen may include any of the elements described above in relation to dosing regimens, such as a dosage, frequency of administration, mode of administration, and duration.

The subject may be any type of subject described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

Treating GI Conditions without Repeating Doses Following Bowel Movements

Embodiments of the invention include methods in which an agent is provided locally to the rectum or colon of a subject, and the agent is not re-administered to the subject following a bowel movement but maintains its therapeutic effect following the bowel movement. Clearance from the colon is a problem with prior methods of administration of therapeutic agents. For example, when 5-ASA is orally administered to subjects, levels of 5-ASA in the colon are decreased by laxative or colonic lavage. De Vos, et al., Concentrations of 5-ASA and Ac-5-ASA in human ileocolonic biopsy homogenates after oral 5-ASA preparations, Gut, 1992 October; 33(10):1338-42, the contents of which are incorporated herein by reference. When 5-ASA is applied topically by enema, colonic 5-ASA decreases dramatically following a bowel movement. Campieri et al., Topical administration of 5-aminosalicylic acid enemas in patients with ulcerative colitis, Studies on rectal absorption and excretion, Gut, 1985, 26, 400-405, the contents of which are incorporated herein by reference.

Methods and compositions of the invention provide stable delivery of therapeutic agents that are retained in the colon even after evacuation of the bowels. Therefore, the invention provides methods in which topical administration of a therapeutic agent does not need to be repeated after the subject moves his bowels.

For example, in certain methods of the invention, efficacy or drug absorption is not hindered or is minimally hindered, i.e., not hindered beyond a defined threshold, after the subject has a bowel movement. In certain methods of the invention, efficacy, drug absorption, and/or drug levels are maintained above a defined threshold after the subject has a bowel movement. Consequently, compared to prior methods, methods of the invention are less burdensome.

A defined amount of the agent may be retained in the colon following a bowel movement. For example, at least 10%, at least 20%, at least 30%, at least 40%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the agent may be retained in the colon following a bowel movement by the subject.

A defined therapeutic effect of the agent may be maintained in the colon following a bowel movement. For example, at least 10%, at least 20%, at least 30%, at least 40%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the therapeutic effect may be maintained in the colon following a bowel movement by the subject.

In methods that do not require re-administration of an agent following a bowel movement, the agent may nonetheless be re-administered at an interval that is independently defined and not dependent on the subject's bowel movements. For example, the agent may be re-administered after one of the intervals described above or according to a dosing regimen described above. The dosing regimen may include any of the elements described above in relation to dosing regimens, such as a dosage, frequency of administration, mode of administration, and duration.

The subject may be any type of subject described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

Treating GI Conditions without Repeating Doses Following Consumption of Food or Liquid

As described below, methods of the invention are also useful for treating conditions of the upper GI tract, such as eosinophilic esophagitis, oral mucositis, and esophageal varices. In certain embodiments, the invention provides methods and compositions that are retained in the upper GI tract, e.g., mouth, esophagus, or stomach, even after consumption of liquids or solid food. Therefore, the invention provides methods in which topical administration of a therapeutic agent does not need to be repeated after the subject eats and/or drinks.

A defined amount of the agent may be retained in the upper GI tract following consumption of food, liquid, or both. For example, at least 10%, at least 20%, at least 30%, at least 40%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the agent may be retained in the upper GI tract following consumption of food, liquid, or both.

A defined therapeutic effect of the agent may be maintained in the upper GI tract following consumption of food, liquid, or both. For example, at least 10%, at least 20%, at least 30%, at least 40%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the therapeutic effect may be maintained in the upper GI tract following consumption of food, liquid, or both.

In methods that do not require re-administration of an agent following consumption of food, liquid, or both, the agent may nonetheless be re-administered at an interval that is independently defined and not dependent on eating or drinking by the subject. For example, the agent may be re-administered after one of the intervals described above or according to a dosing regimen described above. The dosing regimen may include any of the elements described above in relation to dosing regimens, such as a dosage, frequency of administration, mode of administration, and duration.

The subject may be any type of subject described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

Transition from Liquid to Gel

The agent may be provided in a formulation that exists as a liquid when the formulation is below a threshold condition and as a gel when the formulation is above threshold condition. The threshold may be any combination of physical, chemical, and temporal conditions.

The chemical condition may be acidity, alkalinity, or pH. The threshold condition may be a transition pH. The formulation may exist as a liquid when the formulation is below the transition pH and as a gel when the formulation is above the transition pH. The threshold condition may be a transition pH. The formulation may exist as a liquid when the formulation is above the transition pH and as a gel when the formulation is below the transition pH.

The temporal condition may be time. The threshold condition may be a transition time point. The formulation may exist as a liquid prior to the transition time point and as a gel after the transition time point.

The physical condition may be temperature. The threshold condition may be a transition temperature. The formulation may exist as a liquid when the formulation is below the transition temperature and as a gel when the formulation is above the transition temperature. The formulation may exist as a liquid when the formulation is above the transition temperature and as a gel when the formulation is below the transition temperature.

The agent may be provided in a formulation that exists as a liquid at a first temperature and transitions to a gel at a second temperature. The transition from the first temperature to the second temperature may be accompanied by an increase in viscosity. For example, the formulation may exist as a liquid at or near room temperature (about 23° C.) and as a gel at or near physiological temperature (about 37° C.). When such formulations are stored and administered to a patient at room temperature, e.g., via enema, they can readily reach the rectum, sigmoid colon, and descending colon. Once inside the colon, such formulations transition to a gel phase and adhere to the lining of the colon, thereby allowing prolonged exposure of the inflamed tissue to the agent. Formulations that make such phase transitions and their use for delivery of therapeutic agents to the colon are described in, for example, International Patent Publication No. WO 2016/179227; and Sidhartha R. Sinha, et al., A Thermo-Sensitive Delivery Platform for Topical Administration of Inflammatory Bowel Disease Therapies, Gastroenterology, 2015 July; 149(1):52-55.e2, doi: 10.1053/j.gastro.2015.04.002, the contents of each of which are incorporated herein by reference.

For example and without limitation, the formulation may exist as a liquid at about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or about 35° C.

For example and without limitation, the formulation may exist as a liquid at about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

For example and without limitation, the formulation may transition to a gel at from about 16° C. to about 40° C., from about 18° C. to about 40° C., from about 20° C. to about 40° C., from about 22° C. to about 40° C., from about 24° C. to about 40° C., from about 26° C. to about 40° C., from about 28° C. to about 40° C., from about 30° C. to about 40° C., from about 32° C. to about 40° C., from about 34° C. to about 40° C., from about 36° C. to about 40° C., from about 38° C. to about 40° C., from about 16° C. to about 38° C., from about 18° C. to about 38° C., from about 20° C. to about 38° C., from about 22° C. to about 38° C., from about 24° C. to about 38° C., from about 26° C. to about 38° C., from about 28° C. to about 38° C., from about 30° C. to about 38° C., from about 32° C. to about 38° C., from about 34° C. to about 38° C., from about 36° C. to about 38° C., from about 16° C. to about 36° C., from about 18° C. to about 36° C., from about 20° C. to about 36° C., from about 22° C. to about 36° C., from about 24° C. to about 36° C., from about 26° C. to about 36° C., from about 28° C. to about 36° C., from about 30° C. to about 36° C., from about 32° C. to about 36° C., from about 34° C. to about 36° C., from about 16° C. to about 34° C., from about 18° C. to about 34° C., from about 20° C. to about 34° C., from about 22° C. to about 34° C., from about 24° C. to about 34° C., from about 26° C. to about 34° C., from about 28° C. to about 34° C., from about 30° C. to about 34° C., from about 32° C. to about 34° C., from about 16° C. to about 32° C., from about 18° C. to about 32° C., from about 20° C. to about 32° C., from about 22° C. to about 32° C., from about 24° C. to about 32° C., from about 26° C. to about 32° C., from about 28° C. to about 32° C., from about 30° C. to about 32° C., from about 16° C. to about 30° C., from about 18° C. to about 30° C., from about 20° C. to about 30° C., from about 22° C. to about 30° C., from about 24° C. to about 30° C., from about 26° C. to about 30° C., from about 28° C. to about 30° C., from about 16° C. to about 28° C., from about 18° C. to about 28° C., from about 20° C. to about 28° C., from about 22° C. to about 28° C., from about 24° C. to about 28° C., from about 26° C. to about 28° C., from about 16° C. to about 26° C., from about 18° C. to about 26° C., from about 20° C. to about 26° C., from about 22° C. to about 26° C., from about 24° C. to about 26° C., from about 16° C. to about 24° C., from about 18° C. to about 24° C., from about 20° C. to about 24° C., from about 22° C. to about 24° C., from about 16° C. to about 22° C., from about 18° C. to about 22° C., from about 20° C. to about 22° C., from about 16° C. to about 20° C., or from about 18° C. to about 20° C.

The agent may be provided in a formulation that transitions between a liquid phase and a gel phase in response to a stimulus other than, or in addition to, a change in temperature. For example, and without limitation, the stimulus may be or include one or more of a change in pH, solvent exchange, electromagnetic radiation (e.g., visible light, ultraviolet, infrared, X-rays, fluorescence), sound (e.g., ultrasound), pressure, or the presence of specific ions or molecules. Examples of systems that exhibit in situ sol-gel transitions are known in the art and described in, for example, Kouchak, M., In Situ Gelling Systems for Drug Delivery, Jundishapur J Nat Pharm Prod. 2014 August; 9(3): e20126, PMCID: PMC4165193; PMID: 25237648; and Jones and Steed, Gels with sense: supramolecular materials that respond to heat, light and sound, Chem. Soc. Rev., 2016, 45, 6546-6596, DOI 10.1039/C6CS00435K, the contents of each of which are incorporated herein by reference.

### GI Conditions

Methods of the invention may be used to treat any GI condition. For example and without limitation, the GI condition may be achalasia, Barrett's esophagus, Boerhaave syndrome, celiac disease, constipation, Crohn's disease, diverticulitis, enteritis, enterocolitis, eosinophilic esophagitis, esophageal burns, esophageal candidiasis, esophageal spasm, esophageal stricture, esophageal webbing, esophageal varices, esophagitis, gastritis, gastritis, gastroenteritis, gastroesophageal reflux disease, gastrointestinal bleeding, indeterminate colitis, inflammatory bowel disease, intestinal graft-versus-host disease, irritable bowel syndrome, Mallory-Weiss tears, microscopic colitis, nutcracker esophagus, oral mucositis, pernicious anemia, pouchitis, radiation colitis, radiation esophagitis, radiation proctitis, ulcerative colitis, ulcers, or Zenker's diverticulum.

Certain methods of the invention are useful for treatment of IBD. IBD is a group of debilitation conditions, including Crohn's disease, ulcerative colitis, and indeterminate colitis. IBD occurs when tissue in the GI tract becomes inflamed. Crohn's disease may affect tissue of the mouth, esophagus, stomach, small intestine, large intestine, or anus. Ulcerative colitis primarily affects the colon and the rectum. Inflammation may be localized or concentrated in one or more specific parts of the colon, such as the ascending colon, transverse colon, descending colon, sigmoid colon, or the rectum. Indeterminate colitis includes colitis that is deemed not to be either Crohn's disease or ulcerative colitis and may have some features of either or both. IBD may be accompanied by one or more symptoms, such as abdominal pain, anemia arthritis, bronchiolitis obliterans organizing pneumonia, cramps/muscle spasms, deep vein thrombosis (DVT), diarrhea, fatigue, fever, loss of appetite, non-thyroidal illness syndrome (NTIS), primary sclerosing cholangitis, pyoderma gangrenosum, erythema nodosum, arthritis, and rectal bleeding.

Treatment of IBD typically involves two phases. In the first phase (induction), the goal of treatment is to induce remission of the inflammation and provide relief from symptoms. Induction treatment is used during an acute phase or flare of IBD. Once remission has been achieved, the second phase (maintenance) of treatment is directed toward maintaining remission and preventing relapse. Maintenance therapy is generally long-term and continues even when the patient does not experience symptoms. Thus, maintenance therapy is used to maintain the IBD in a reduced state. Induction and maintenance phases may involve the same or different medications, modes of administration, frequencies of administration, and dosages.

An acute phase of IBD may have one or more of the following features described above in relation to acute phases of conditions generally. As indicated above, acute phases of IBD are sometimes called as “flare-ups”. The following markers may be used to diagnose IBD, determine its level of severity, and characterize the phase of the condition, e.g., determine whether it is acute or non-acute: albumin, anti-neutrophil cytoplasmic antibody (ANCA), anti-Saccharomyces cerevisiae antibodies (ASCA), C reactive protein (CRP), calprotectin, erythrocyte sedimentation rate (ESR), lactoferrin, leucocyte count, platelet count, and al acid glycoprotein (orosomucoid). IBD can also be evaluated by analysis of proteome, transcriptome, genome, and post-translational modifications, such as phosphorylation, acetylation, glycosylation, disulfide bond formation, deamidation, and citrullination. Biomarkers of IBD and their diagnostic application are described in more detail in, for example, Bennike T. et al., Biomarkers in inflammatory bowel diseases: Current status and proteomics identification strategies, World J Gastroenterol. 2014 Mar. 28; 20(12): 3231-3244, doi: 10.3748/wjg.v20.i12.3231; Mohsen Norouzinia et al., Biomarkers in inflammatory bowel diseases: insight into diagnosis, prognosis and treatment, Gastroenterol Hepatol Bed Bench, 2017 Summer; 10(3): 155-167; and Viennois E., et al., Biomarkers of IBD: from classical laboratory tools to personalized medicine, Inflamm Bowel Dis. 2015 October; 21(10): 2467-2474, doi: 10.1097/MIB.0000000000000444, the contents of each of which are incorporated herein by reference.

Methods of the invention are also useful for treatment of irritable bowel syndrome (IBS).

IBS comprises GI symptoms, such as abdominal pain and changes in the pattern of bowel movements, without any evidence of underlying damage. Symptoms may occur over a period of years. IBS has been classified into the following four types based on whether diarrhea and constipation are common: IBS-D, in which diarrhea is common; IBS-C, in which constipation is common; IBS-M, in which both diarrhea and constipation are common; and IBS-U, in which neither diarrhea nor constipation is common.

Methods of the invention may also be used to treat conditions of the upper GI tract. For example and without limitation, methods may include treatment of eosinophilic esophagitis, oral mucositis, or esophageal varices.

### Examples

### Two Compartments

During the stability of single compartment formulation, most of the formulations showed elevation in gelation temperature (>40 C). In addition to this, the change in description is also observed. The description changes from light pink dispersion to brownish dispersion. In order to address the challenges, two compartment formulations were hypothesized. One compartment will contain API and the other diluent. The API needs to be reconstituted prior to usage.

The target for the 2 compartment formulations:
Gelation temperature 30-37 C
Gelation time—less than 5 minutes
Reconstitution time—less than 1 minutes
Viscosity at RT—less than 3000 cps
Reconstituted formulation should be stable for at least 3 hours

Based on the excipients used in the formulation, it has been classified into non lipid and lipid based formulation.

### Non Lipid Based Formulation

During the formulation development, we have observed one diluent formulation doesn't gel upon heating to 50 C, however it starts gelling only after reconstitution with Mesalamine. The composition is provided as below:

TABLE 1
composition for non-lipid formulation

| Batch no | POC-0718/242/P |
|---|---|
| Poloxamer 407 | 13.5 |
| PVP (Kollidon 30) | 1 |
| Polycarbophil | 0.25 |
| Edetate disodium | 0.1 |
| NaCl | 0.5 |
| Vitamin E TPGS | 0.2 |
| Transcutol | 10 |
| Tris | 0.15 |
| Purified water QS | 93.33 |
| to | |

### Observation

The initial gelation temperature is satisfactory.

### Stability Batches:

To understand the reproducibility of the gelation temperature, multiple batches were prepared and stability was monitored. In addition to this in one of batch, source of Mesalamine is varied to understand its impact on the various physical parameters.

The compositions for the stability batches are provided below:

TABLE 2
Composition for stability batches of Non-formulation

| Batch no | POC-0718/242/P | POC-0718/354/P | POC-0718/472/P | POC-0718/516/P | POC-0718/557/P |
|---|---|---|---|---|---|
| Poloxamer 407 | 13.5 | 13.5 | 13.5 | 13.5 | 13.5 |
| PVP (Kollidon | 1 | 1 | 1 | 1 | 1 |
| 30) | | | | | |
| Polycarbophil | 0.25 | 0.25 | 0.25 | 0.25 | 0.25 |
| Edetate | 0.1 | 0.1 | 0.1 | 0.1 | 0.1 |
| disodium | | | | | |
| NaCl | 0.5 | 0.5 | 0.5 | 0.5 | 0.5 |
| Vitamin E | 0.2 | 0.2 | 0.2 | 0.2 | 0.2 |
| TPGS | | | | | |
| Transcutol | 10 | 10 | 10 | 10 | 10 |
| Tris | 0.15 | 0.15 | 0.15 | 0.15 | 0.15 |
| Purified water | 93.33 | 93.33 | 93.33 | 93.33 | 93.33 |
| QS to | | | | | |
| Source of | MSN | Cambrex | | | |
| Mesalamine | | | | | |
| Grade of | Micronized | S | | | |
| Mesalamine | | | | | |
| MOC of | | | | | |
| packaging | | | | | |
| material | | | | | |
| Tgel (deg C.) | >40 | >40 | >40 | >40 | >40 |
| diluent | | | | | |
| T gel (deg C.) | | | | | |
| Reconstituted | | | | | |

### Manufacturing Process:

Manufacturing of non-lipid diluent comprises of multiple steps. A detailed manufacturing process is provided below:

### Preparation of Tris Solution:

Tris was dissolved in part quantity of water under stirring. Stirring was continued till a clear colorless solution is observed.

### Preparation of Polycarbophil Phase:

Polycarbophil was dispersed in part quantity of purified water under stirring. The stirring was continued till a lump free dispersion is observed. The Tris solution prepared above is added slowly to the polycarbophil dispersion under stirring. Stirring was continued for 15 minutes.

### Preparation of Sodium Chloride Solution:

Weighed quantity of Sodium chloride was dissolved in part quantity of purified water.

### Preparation of the Diluent:

Purified water taken in a stainless-steel container and heated to 50±5 C.
To it added and dissolved Edetate disodium and Vitamin E TPGS under stirring.
Cooled the solution to 2-5 C using an ice water bath.
Poloxamer 407 was added under vortex for a uniform dispersion. Stirring was continued while maintaining the temperature (not more than 5 C) till complete dissolution of Poloxamer 407.
To this added required quantity of Transcutol P under stirring. Stirring was further continued for another 15 minutes for uniform mixing.
To this mixture previously prepared Polycarbophil phase was added under stirring while maintaining the product temperature less than 5 C.
To this mixture previously prepared Sodium chloride solution was added under stirring while maintaining the product temperature less than 5 C.
The mixture was removed from ice water bath and stirring was continued till the product temperature reaches to 25 C
To this PVP (Kollidon 30) was added under stirring. Stirring was continued till complete solubilization of PVP.

### Stability Data:

| Batch | | | | Assay of | Single | Total |
|---|---|---|---|---|---|---|
| no | Condition | Tg | pH | Mesalamine | max | impurities |
| POC- | Initial | NA | NA | NA | NA | NA |
| 0718/ | 1 M, 25/60 | 31-32 | 5.00 | 99.8 | NA | NA |
| 242/P | 2 M, 25/60 | 31-32 | 4.94 | 101.4 | NA | NA |
| | 3 M, 25/60 | 32-33 | 4.97 | NA | 0.03 | 0.08 |
| | 6 M, 25/60 | 33-34 | 5.04 | 100.3 | 0.03 | 0.06 |
| | 9 M, 25/60 | 33-34 | 5.02 | NA | 0.02 | 0.09 |
| | 12 M, 25/60 | 33-34 | 4.98 | NA | 0.04 | 0.07 |
| | 3 M, 30/65 | 32-33 | 5.00 | NA | 0.02 | 0.05 |
| | 6 M, 30/65 | 33-34 | 5.03 | 92.8 | NA | NA |
| | 9 M, 30/65 | 33-34 | 5.02 | NA | 0.02 | 0.07 |
| | 12 M, 30/65 | 33-34 | 4.99 | NA | 0.02 | 0.05 |
| | 1 M, 40/75 | 31-32 | 5.00 | 100.3 | NA | NA |
| | 2 M, 40/75 | 30-31 | 4.93 | 100.4 | NA | NA |
| | 3 M, 40/75 | 32-33 | 4.96 | NA | 0.01 | 0.03 |
| | 6 M, 40/75 | 33-34 | 5.02 | 97.5 | 0.03 | 006 |
| POC- | Initial | 29-30 | 5.07 | 99.4 | 0.34 | 0.11 |
| 0718/ | 1 M, 25/60 | 31-32 | 5.05 | 100.7 | 0.33 | 0.11 |
| 354/P | 2 M, 25/60 | 32-33 | 5.00 | NA | 0.01 | 0.01 |
| | 3 M, 25/60 | 29-30 | 4.99 | 101.4 | 0.02 | 0.04 |
| | 6 M, 25/60 | 35-36 | 5.08 | NA | NA | NA |
| | 9 M, 25/60 | 30-31 | 5.10 | NA | NA | NA |
| | 1 M, 40/75 | 31-32 | 5.06 | 102.6 | 0.32 | 0.10 |
| | 2 M, 40/75 | 32-33 | 5.00 | NA | 0.01 | 0.01 |
| | 3 M, 40/75 | 29-30 | 4.98 | 101.1 | 0.02 | 0.02 |
| | 6 M, 40/75 | 34-35 | 5.07 | NA | NA | NA |
| POC- | Initial | 30-31 | 4.94 | NA | NA | NA |
| 0718/ | 1 M, 25/60 | 33-34 | 5.04 | NA | NA | NA |
| 472/P | 2 M, 25/60 | 32-33 | 5.08 | NA | NA | NA |
| | 3 M, 25/60 | 32-33 | 5.07 | NA | NA | NA |
| | 1 M, 40/75 | 33-34 | 5.04 | NA | NA | NA |
| | 2 M, 40/75 | 32-33 | 5.07 | NA | NA | NA |
| | 3 M, 40/75 | 32-33 | 5.10 | NA | NA | NA |

### Observation:

No significant change in gelation temperature and pH is observed in all batches. Formulation is found to be stable up to 12 months. Changes in excipients of non-lipid formulations Further changes in the excipient concentration were carried out to understand the impact on gelation temperature. The compositions are presented as below:

TABLE 3
composition of Non-lipid formulation

| | POC- | POC- | POC- | POC- | POC- | POC- | POC- | POC- | POC- | POC- | POC- | POC- | POC- | POC- |
|---|---|---|---|---|---|---|---|---|---|---|---|---|---|---|
| | 0718/ | 0718/ | 0718/ | 0718/ | 0718/ | 0718/ | 0718/ | 0718/ | 0718/ | 0718/ | 0718/ | 0718/ | 0718/ | 0718/ |
| Batch no | 284/P | 285/P | 400/P | 401/P | 478/P | 488/P | 489/P | 490/P | 499/P | 500/P | 509/P | 511/P | 512/P | 513/P |
| Poloxamer 407 | 13.5 | 13.5 | 13 | 13.25 | 13.5 | 13.5 | 13.5 | 13.5 | 13.5 | 13.5 | 13.5 | 13.5 | 13.5 | 13.5 |
| PVP | 1.5 | 1.25 | 1 | 1 | 1 | 1 | 1 | 1 | | 1 | | 1 | 1 | 1 |
| (Kollidon 30) | | | | | | | | | | | | | | |
| Polycarbophil | 0.25 | 0.25 | 0.25 | 0.25 | | 0.25 | 0.25 | 0.25 | 0.25 | | 0.25 | 0.25 | 0.25 | 0.25 |
| Edetate | 0.1 | 0.1 | 0.1 | 0.1 | 0.1 | | | | | | | 0.1 | 0.1 | 0.1 |
| disodium | | | | | | | | | | | | | | |
| NaCl | 0.5 | 0.5 | 0.5 | 0.5 | 0.5 | 0.5 | | | | | 0.5 | 0.75 | 1 | |
| Vitamin E | 0.2 | 0.2 | 0.2 | 0.2 | 0.2 | 0.2 | 0.2 | | | | | 0.2 | 0.2 | 0.2 |
| TPGS | | | | | | | | | | | | | | |
| Transcutol | 10 | 10 | 10 | 10 | 10 | 10 | 10 | 10 | 10 | 10 | 10 | 10 | 10 | 10 |
| Tris | 0.15 | 0.15 | 0.15 | 0.15 | | 0.15 | 0.15 | 0.15 | 0.15 | | 0.15 | 0.15 | 0.15 | 0.15 |
| Purified | 93.33 | 93.33 | 93.33 | 93.33 | 93.33 | 93.33 | 93.33 | 93.33 | 93.33 | 93.33 | 93.33 | 93.33 | 93.33 | 93.33 |
| water QS to | | | | | | | | | | | | | | |
| Tgel (deg C.) | >40 | >40 | >40 | >40 | >40 | 35-36 | 34-35 | 33-34 | 29-30 | >40 | 26-27 | >40 | >40 | 33-34 |

### Manufacturing Process:

POC-0718/285/P, POC-0718/400/P, POC-0718/401/P

### Preparation of Poloxamer Phase:

In part quantity of purified water, Edetate disodium was dissolved
Heated to 55° C. Dissolved Vitamin E TPGS under stirring at 200-400 RPM
Cooled to 5±3° C. Added Poloxamer 407 under stirring for 3 hours
Stored in cooling cabinet maintained at 5±3° C. overnight

### Preparation of Tris Solution

Tris was dissolved in part quantity of water

### Preparation of Polycarbophil Phase

Polycarbophil was dispersed in part quantity of water under stirring at 500-600 RPM
Tris solution added to the above dispersion under stirring at 500±200 RPM Preparation of Sodium chloride solution
Sodium chloride was dissolved in part quantity of water Processing in the main manufacturing vessel
Transcutol P was added to the Poloxamer phase. Mixed for 15 minutes at 750±200 RPM
Polycarbophil phase was added to the above mixture. Mixed for 15 minutes at 750±200 RPM
Sodium chloride solution was added to the above mixture. Mixed for 15 minutes at 750±200 RPM
Stirring continued without ice bath to raise the product temperature
It took approximately 1.5 hours to reach the temperature of 25° C.
Kolliodon 30 was added to it. Stirred for 2 hours at 75±200 RPM

### POC-0718/478/P

### Preparation of Tris Solution

Tris was dissolved in part quantity of water
Preparation of Sodium chloride solution
Sodium chloride was dissolved in part quantity of water

### Preparation of Poloxamer Phase:

In part quantity of purified water, Edetate disodium was dissolved
Heated to 55° C. Dissolved Vitamin E TPGS under stirring at 200-400 RPM
Cooled to 5±3° C. Added Poloxamer 407 under stirring. Stirred for 3 hours

### Processing in the Main Manufacturing Vessel

Transcutol P was added to the Poloxamer phase. Mixed for 15 minutes at 750±200 RPM
Sodium chloride solution was added to the above mixture. Mixed for 15 minutes at 750±200 RPM
Stirring continued without ice bath to raise the product temperature
It took approximately 1.5 hours to reach the temperature of 25° C.
Kolliodon 30 was added to it. Stirred for 2 hours at 75±200 RPM

### POC-0718/488/P

### Preparation of Tris Solution

Tris was dissolved in part quantity of water

### Preparation of Polycarbophil Phase

Polycarbophil was dispersed in part quantity of water under stirring at 500-600 RPM
Tris solution added to the above dispersion under stirring at 500±200 RPM

### Preparation of Poloxamer Phase:

Part quantity of water heated to 55° C. Dissolved Vitamin E TPGS under stirring at 200-400 RPM
Cooled to 5±3° C. Added Poloxamer 407 under stirring. Stirring continued for 3 hours Preparation of Sodium chloride solution
Sodium chloride was dissolved in part quantity of water

### Processing in the Main Manufacturing Vessel

Transcutol P was added to the Poloxamer phase. Mixed for 15 minutes at 750±200 RPM
Polycarbophil phase was added to the above mixture. Mixed for 15 minutes at 750±200 RPM
Sodium chloride solution was added to the above mixture. Mixed for 15 minutes at 750±200 RPM
Stirring continued without ice bath to raise the product temperature
It took approximately 1.5 hours to reach the temperature of 25° C.
Kolliodon 30 was added to it. Stirred for 2 hours at 75±200 RPM

### POC-0718/489/P

### Preparation of Tris Solution

Tris was dissolved in part quantity of water Preparation of Polycarbophil phase
Polycarbophil was dispersed in part quantity of water under stirring at 500-600 RPM
Tris solution added to the above dispersion under stirring at 500±200 RPM Preparation of Poloxamer Phase:
Part quantity of water heated to 55° C. Dissolved Vitamin E TPGS under stirring at 200-400 RPM
Cooled to 5±3° C. Added Poloxamer 407 under stirring. Stirring continued for 3 hours Processing in the main manufacturing vessel
Transcutol P was added to the Poloxamer phase. Mixed for 15 minutes at 750±200 RPM
Polycarbophil phase was added to the above mixture. Mixed for 15 minutes at 750±200 RPM
Stirring continued without ice bath to raise the product temperature
It took approximately 1.5 hours to reach the temperature of 25° C.
Kolliodon 30 was added to it. Stirred for 2 hours at 75±200 RPM

### POC-0718/490/P

### Preparation of Tris Solution

Tris was dissolved in part quantity of water

### Preparation of Polycarbophil Phase

Polycarbophil was dispersed in part quantity of water under stirring at 500-600 RPM
Tris solution added to the above dispersion under stirring at 500±200 RPM

### Preparation of Poloxamer Phase:

Part quantity of water cooled to 5±3° C. Added Poloxamer 407 under stirring. Stirring continued for 3 hours

### Processing in the Main Manufacturing Vessel

Transcutol P was added to the Poloxamer phase. Mixed for 15 minutes at 750±200 RPM
Polycarbophil phase was added to the above mixture. Mixed for 15 minutes at 750±200 RPM
Stirring continued without ice bath to raise the product temperature
It took approximately 1.5 hours to reach the temperature of 25° C.
Kolliodon 30 was added to it. Stirred for 2 hours at 75±200 RPM

### POC-0718/499/P

### Preparation of Tris Solution

Tris was dissolved in part quantity of water

### Preparation of Polycarbophil Phase

Polycarbophil was dispersed in part quantity of water under stirring at 500-600 RPM
Tris solution added to the above dispersion under stirring at 500±200 RPM

### Preparation of Poloxamer Phase:

Part quantity of water cooled to 5±3° C. Added Poloxamer 407 under stirring. Stirring continued for 3 hours

### Processing in the Main Manufacturing Vessel

Transcutol P was added to the Poloxamer phase. Mixed for 15 minutes at 750±200 RPM
Polycarbophil phase was added to the above mixture. Mixed for 15 minutes at 750±200 RPM
Stirring continued without ice bath to raise the product temperature
It took approximately 1.5 hours to reach the temperature of 25° C.

### POC-0718/511/P, POC-0718/512/P

### Preparation of Poloxamer Phase:

In part quantity of purified water, Edetate disodium was dissolved
Heated to 55° C. Dissolved Vitamin E TPGS under stirring at 200-400 RPM
Cooled to 5±3° C. Added Poloxamer 407 under stirring
Stirred for 3 hours till complete dissolution of Poloxamer

### Preparation of Tris Solution

Tris was dissolved in part quantity of water

### Preparation of Polycarbophil Phase

Polycarbophil was dispersed in part quantity of water under stirring at 500-600 RPM
Tris solution added to the above dispersion under stirring at 500±200 RPM

### Preparation of Sodium Chloride Solution

Sodium chloride was dissolved in part quantity of water Processing in the main manufacturing vessel
Transcutol P was added to the Poloxamer phase. Mixed for 15 minutes at 750±200 RPM
Polycarbophil phase was added to the above mixture. Mixed for 15 minutes at 750±200 RPM
Sodium chloride solution was added to the above mixture. Mixed for 15 minutes at 750±200 RPM
Stirring continued without ice bath to raise the product temperature
It took approximately 1.5 hours to reach the temperature of 25° C.
Kolliodon 30 was added to it. Stirred for 2 hours at 75±200 RPM

### POC-0718/513/P

### Preparation of Poloxamer Phase:

In part quantity of purified water, Edetate disodium was dissolved
Heated to 55° C. Dissolved Vitamin E TPGS under stirring at 200-400 RPM
Cooled to 5±3° C. Added Poloxamer 407 under stirring
Stirred for 3 hours till complete dissolution of Poloxamer

### Preparation of Tris Solution

Tris was dissolved in part quantity of water

### Preparation of Polycarbophil Phase

Polycarbophil was dispersed in part quantity of water under stirring at 500-600 RPM
Tris solution added to the above dispersion under stirring at 500±200 RPM

### Processing in the Main Manufacturing Vessel

Transcutol P was added to the Poloxamer phase. Mixed for 15 minutes at 750±200 RPM
Polycarbophil phase was added to the above mixture. Mixed for 15 minutes at 750±200 RPM
Stirring continued without ice bath to raise the product temperature
It took approximately 1.5 hours to reach the temperature of 25° C.
Kolliodon 30 was added to it. Stirred for 2 hours at 75±200 RPM

### Observation:

Any minute changes in the composition leads to variation in gelation temperature.

### Effect of Mesalamine Concentration on the Gelation Temperature

Different concentration of mesalamine was dispersed in the non-lipid diluent to understand its impact on the gelation temperature

| | | |
|---|---|---|
| Weight of | Placebo | |
| Mesalamine | (POC-0718/472/P) | Tg (° C.) |

| | | | | |
|---|---|---|---|---|
| 0.1 | gm | 59.9 | gm | Slightly thicken at |
| | | | | 32-33 but Tg >40. |
| 0.2 | gm | 59.8 | gm | 36-37 |
| 0.3 | gm | 59.7 | gm | 32-33 |
| 0.4 | gm | 59.6 | gm | 32-33 |
| 0.5 | gm | 59.5 | gm | 32-33 |
| 1 | gm | 59.0 | gm | 32-33 |
| 2 | gm | 58.0 | gm | 32-33 |
| 6 | gm | 54.0 | gm | 32-33 |

### Observation:

0.3 g and higher amount of Mesalamine shows desired gelation temperature.

### Lipid Formulation

The gelation phenomenon of Poloxamer is reversible and characterized by a sol-gel transition temperature. The thermogelation is due to hydrophobic interactions between the poloxamer 407 copolymer chains. By elevating the temperature, the poloxamer 407 copolymer chains start to aggregate into a micellar structure. The formation of micelle structures is a result of the dehydration of the hydrophobic PPO repeat units and defines the initial step of gelation. Phospholipids are being widely used in formation of micelle.

However the phospholipids are hard to stabilize in an aqueous environment. The long term stability or shelf-life of a drug product containing lipids can be dramatically affected by the lipid species used in the formulation. The most degradation pathway is oxidation and hydrolysis¹²³. Generally, the more unsaturated a compound, the easier the product is oxidized, and thus the shorter the shelf life of the product. Lipids from biological sources (e.g., egg, bovine, or soybean) typically contain significant levels of polyunsaturated fatty acids and therefore are inherently less stable than their synthetic counterparts. While saturated lipids offer the greatest stability in terms of oxidation, they also have much higher transition temperatures and thus present other difficulties in formulation.

Aqueous formulations of drug products tend to be less stable since the presence of excess or bulk water leads to rapid hydrolytic degradation in lipid preparations. This hydrolysis is dependent on several factors including pH, temperature, buffer species, ionic strength, acyl chain length and headgroup, and the state of aggregation. ¹Frrkjaer, S., Hjorth, E. L., and Wrrts, O., Stability and storage of liposomes, in Optimization of Drug Delivery, Bundgaard, H., Bagger Hansen, A., and Kofod, H., Eds., Munksgaard, Copenhagen, 1982, 384.²Kensil, C. R. and Dennis, E. A., Alkaline hydrolysis of phospholipids in model membranes and the dependence on their state of aggregation, Biochemistry, 20, 6079, 1981.³ Grit, M., de Smidt, J. H., Struijke, A., and Crommelin, D. J. A., Hydrolysis of phosphatidylcholine in aqueous liposome dispersions, Int. J. Pharm., 50, 1, 1989.

Various lipids of saturated and unsaturated nature were screened. The details of the lipid screened are as below:

### Saturated Lipid

Phospholipon 90 H
DSPC
DPPC
Lipoid SPC 3

### Unsaturated Lipid

Lipoid S 100
Phospholipon 90 G
   Formulation with Phospholipon 90 H

Lipoid 90 H is a saturated lipid contains not less than 90% of hydrogenated phosphatidylcholine. Different concentration of Phospholipon 90H and Poloxamer 407 were screened and the compositions are presented in Table 4.

TABLE 4
Composition for formulation with 90H (Ref Jul. 21, 2020)

| | POC- | POC- | POC- | POC- | POC- | POC- | POC- |
|---|---|---|---|---|---|---|---|
| | 0718/ | 0718/ | 0718/ | 0718/ | 0718- | 0718- | 0718- |
| Batch no | 271/P | 281/P | 282/P | 286/P | 287/P | 305/P | 307/P |
| Poloxamer 407 | 12.5 | 12.5 | 12 | 13 | 13.5 | 13.5 | 13.5 |
| Edetate disodium | 0.1 | 0.1 | 0.1 | 0.1 | 0.1 | 0.1 | 0.1 |
| Transcutol | 10 | 10 | 10 | 10 | 10 | 10 | 10 |
| Phospholipon 90 H | 0.5 | 2 | 2 | 2 | 2 | 1 | 1 |
| Purified water QS to | 93.33 | 93.33 | 93.33 | 93.33 | 93.33 | 93.33 | 93.33 |
| Tgel (deg C.) | 40 | 40 | 40 | 32.5 | 29.5 | 32.5 | 32.5 |

### Manufacturing Process:

### POC-0718/271/P:

Poloxamer phase
   Edetate disodium was dissolved in purified water
   The solution was cooled to 5±3 C
   Poloxamer added to the cooled solution under stirring. Stirred at 400-600 RPM for 2 hours
   Kept in cooling cabinet overnight for complete solubilization of Poloxamer
Lipid phase
   Phospholipon 90 H dissolved in transcutol at 45-50 C
Emulsification
   Poloxamer phase was heated to 45 C
   Lipid phase added to Poloxamer phase under stirring
   Homogenized at 6000 RPM for 30 minutes
   Cooled to room temperature under stirring

### POC-0718/282/P, POC-0718/286/P, POC-0718/287/P:

Poloxamer phase
   Edetate disodium was dissolved in purified water
   The solution was cooled to 5±3 C
   Poloxamer added to the cooled solution under stirring. Stirred at 400-600 RPM for 2 hours
   Kept in cooling cabinet overnight for complete solubilization of Poloxamer
Lipid phase
   Phospholipon 90 H dissolved in transcutol at 45-50 C
Emulsification
   Poloxamer phase was heated to 45-50° C.
   Lipid phase added to Poloxamer phase under stirring
   Homogenized at 7500 RPM for 15 minutes
   Cooled to room temperature under stirring

### Stress Study:

Among all the batches, #286, #305 and #307 showed desired gelation temperature. Reduction in gelation temperature is observed with increase in Poloxamer concentration (13% and above) All these batches were subjected to stress study at 60 C. at the end of 1 week all formulations showed gelation temperature of more than 40 C

### Observation:

Phospholipon 90H has transition temperature of 55 C. So the emulsification temperature needs to be kept at 40-50 C. At the higher temperature the Poloxamer phase starts thickening which leads to foam generation.

All the formulations showed non gelation behavior at the end of lweek at 60 C. Considering this the strategy was discontinued.

### Formulation with Lipoid SPC 3

Lipoid SPC 3 is a saturated phospholipid and shows better aqueous stability when compared with the unsaturated phospholipids. It is a saturated phospholipid with lower iodine value (not more than 3). The lower the iodine value, the lower is the unsaturation.

Lipoid SPC shows limited solubility in Transcutol P at room temperature and shows physical instability for the formulation made at room temperature. For improved physical stability, medium chain triglyceride has been used in few of the experiments as solvent to solubilize Lipoid SPC 3.

The compositions for the formulation with Lipoid SPC 3 are described as below:

TABLE 5
Composition for formulation containing Lipoid SPC 3

| | POC- | POC- | POC- | POC- | POC- | POC- | POC- |
|---|---|---|---|---|---|---|---|
| | 0718/ | 0718/ | 0718/ | 0718/ | 0718/ | 0718/ | 0718/ |
| Batch no | 426/P | 427/P | 428/P | 433/P | 434/P | 435/P | 436/P |
| Poloxamer 407 | 13 | 12 | 12.5 | 12 | 11 | 12 | 12 |
| Edetate disodium | 0.1 | 0.1 | 0.1 | 0.1 | 0.1 | 0.1 | 0.1 |
| Transcutol | 10 | 5 | 10 | 5 | 5 | 5 | 5 |
| MCT (Migylol 812) | — | 10 | — | 5 | 10 | 5 | 5 |
| Lipoid SPC 3 | 1 | 1 | 1 | 1 | 1 | 0.5 | 0.75 |
| Purified water QS to | 93.33 | 93.33 | 93.33 | 93.33 | 93.33 | 93.33 | 93.33 |
| Tgel (deg C.) | 32-33 | 27-28 | >40 | 30-31 | 33-34 | 30-31 | 29-30 |
| pH | 6.09 | 6.01 | | 7.04 | 7.06 | 7.1 | 7.11 |

### Manufacturing Process for POC-0718/426:

Poloxamer phase
   Edetate disodium was dissolved in purified water
   The solution was cooled to 5±3 C
   Poloxamer added to the cooled solution under stirring. Stirred at 400-600 RPM for 2 hours
   Kept in cooling cabinet overnight for complete solubilization of Poloxamer
Lipid phase
   Lipoid SPC 3 was dissolved in transcutol at 40 C
Emulsification
   Poloxamer phase was brought to RT under stirring
   Lipid phase added to Poloxamer phase under stirring
   Homogenized at 9000 RPM for 30 minutes
   Emulsion with few Lipoid particles were observed at the surface of the formulation

### Manufacturing Process for POC-0718/427:

Poloxamer phase
   Edetate disodium was dissolved in purified water
   The solution was cooled to 5±3 C
   Poloxamer added to the cooled solution under stirring. Stirred at 400-600 RPM for 2 hours
   Kept in cooling cabinet overnight for complete solubilization of Poloxamer
Lipid phase
   Lipoid SPC 3 was dissolved in transcutol and medium chain triglyceride at 55-60 C
Emulsification
   Poloxamer phase was brought to RT under stirring
   Lipid phase added to Poloxamer phase under stirring
   Homogenized at 9000 RPM for 30 minutes

Few formulations were made without edetate disodium to understand the impact on the gelation temperature. The formulation details are presented in Table 6.

TABLE 6
Composition for Lipoid SPC 3 formulation without Edetate disodium

| Batch no | POC-0718/437/P | POC-0718/438/P | POC-0718/439/P | POC-0718/440/P | POC-0718/441/P |
|---|---|---|---|---|---|
| Poloxamer 407 | 11 | 11.25 | 11.5 | 12 | 10.75 |
| Transcutol | 5 | 5 | 5 | 5 | 5 |
| MCT (Migylol | 5 | 10 | 10 | 10 | 10 |
| 812) | | | | | |
| Lipoid SPC 3 | 1 | 1 | 1 | 1 | 1 |
| Purified water QS | 93.33 | 93.33 | 93.33 | 93.33 | 93.33 |
| to | | | | | |
| Tgel (deg C.) | >40 | 31-32 | 29-30 | 27-28 | >40 |
| pH | | 7.03 | 7.1 | | |

### Manufacturing Process for POC-0718/437:

Poloxamer phase
   Purified water was cooled to 5±3 C
   Poloxamer added to the cooled solution under stirring. Stirred at 400-600 RPM for 2 hours
   Kept in cooling cabinet overnight for complete solubilization of Poloxamer
Lipid phase
   Lipoid SPC 3 was dissolved in transcutol and medium chain triglyceride at 55-60 C
Emulsification
   Poloxamer phase was brought to RT under stirring
   Lipid phase added to Poloxamer phase under stirring
   Homogenized at 9000 RPM for 30 minutes

These formulations were packed in both packaging material (HDPE bottle and EVOh coated HDPE bottle) subjected to stress study (60 C) to access the stability at elevated temperature. During this study, only gelation temperature was monitored as a key test parameter. The results are present below:

### Stability Study:

#426 and #427 showed promising gelation temperature. Reproducible batches were manufactured for stability study. The composition for the stability batches are presented here:

Batch no

| | |
|---|---|
| POC-0718/470/P | POC-0718/471/P |

Reference Batch no

| | POC-0718/426/P | POC-0718/434/P |
|---|---|---|
| Poloxamer 407 | 13 | 11 |
| Edetate | 0.1 | 0.1 |
| disodium | | |
| Transcutol | 10 | 5 |
| MCT (Migylol | — | 10 |
| 812 | | |
| Lipoid SPC 3 | 1 | 1 |
| Purified water | 93.33 | 93.33 |
| QS to | | |
| Tgel (deg C.) | 32-33 | 33-34 |
| pH | 6.09 | 7.06 |

### Manufacturing Process for POC-0718/470:

### Manufacturing Process for POC-0718/471:

During the stability study, only gelation temperature and the pH of the reconstituted formulation was monitored.

| Batch no | Condition | Tg | pH |
|---|---|---|---|
| POC-- | Initial | 32-33 | 4.64 |
| 0718/470/P | 1 M, 25° C./60% RH | 34-35 | 4.72 |
| | 2 M, 25° C./60% RH | 33-34 | 4.75 |
| | 3 M, 25° C./60% RH | 33-34 | 4.78 |
| | 6 M, 25° C./60% RH | >40 | 4.70 |
| | 1 M, 40° C./75% RH | 35-36 | 4.74 |
| | 2 M, 40° C./75% RH | 34-35 | 4.76 |
| | 3M, 40° C./75% RH | 34-35 | 4.79 |
| | 6M, 40° C./75% RH | >40 | 4.71 |
| POC-- | Initial | 33-34 | 4.61 |
| 0718/471/P | 1 M, 25° C./60% RH | 31-32 | 4.77 |
| | 2 M, 25° C./60% RH | 31-32 | 4.76 |
| | 3 M, 25° C./60% RH | 31-32 | 4.79 |
| | 6 M, 25° C./60% RH | 31-32 | 4.65 |
| | 1 M, 40° C./75% RH | 31-32 | 4.72 |
| | 2 M, 40° C./75% RH | 34-35 | 4.55 |
| | 3 M, 40° C./75% RH | 36-37 | 4.54 |
| | 6 M, 40° C./75% RH | >40 | 4.06 |

### Conclusion

Both the formulations showed elevation in gelation temperature at 6M time point. Based on this, the strategy was discontinued.
   Formulation with Lipoid S 100

Lipoid S 100 is an unsaturated phospholipid with lower transition temperature. Multiple experiments were executed to understand the impact of formulation on the gelation temperature. Composition for the prototype formulations are presented below:

TABLE 7
Composition for formulation with Lipoid S 100

| | POC- | POC- | POC- | POC- | POC- | POC- | POC- |
|---|---|---|---|---|---|---|---|
| | 0718/ | 0718/ | 0718/ | 0718/ | 0718/ | 0718/ | 0718/ |
| Ingredients | 188/P | 226/P | 231/P | 235/P | 236/P | 243/P | 265/P |
| Edetate sodium | 0.1 | 0.1 | — | 0.1 | 0.1 | 0.1 | 0.1 |
| Poloxamer 407 | 12 | 12 | 12 | 12 | 12 | 12 | 13 |
| Lipoid s 100 | — | 2.0 | 2.0 | 1.0 | 0.5 | 0.75 | 0.5 |
| Polycarbophil | 0.25 | 0.25 | — | 0.25 | 0.25 | 0.25 | 0.25 |
| NaCl | 0.5 | 0.5 | — | 0.5 | 0.5 | 0.5 | 0.5 |
| TPGS | 0.2 | 0.2 | — | 0.2 | 0.2 | 0.2 | 0.2 |
| Transcutol | 10 | 10 | 10 | 10 | 10 | 10 | 10 |
| Tris | 0.15 | 0.15 | — | 0.15 | 0.15 | 0.15 | 0.15 |
| Purified water | QS to | QS to | QS to | QS to | QS to | QS to | QS to |
| | 93.33 | 93.33 | 93.33 | 93.33 | 93.33 | 93.33 | 93.33 |
| Tg (° C.) | >40 | 25-26 | | 28-29 | 35-36 | 28-29 | 24-25 |

Most of the formulations showed gelation temperature in the range of 25-36. In order to understand the impact of heat and time on the gelation temperature, all these formulations were kept at 60 C for 4 weeks. The gelation temperature was monitored every week. The results are presented as below:

TABLE 8
Stress study results for Lipoid S 100 containing formulations

| | | 60° C., | 60° C., | 60° C., | 60° C., |
|---|---|---|---|---|---|
| Batch no | Initial | 7 days | 14 days | 21 days | 28 days |
| POC-0718/231/P | 34 | 27.5 | 26.5 | 29.5 | 35 |
| POC-0718/236/P | 35.5 | 40 | 40 | 40 | 40 |
| POC-0718/243/P | 28.5 | 40 | 40 | 40 | 40 |
| POC-0718/235/P | 28.5 | 25.5 | 25.5 | 29.5 | 28.5 |
| POC-0718/265/P | 24.5 | 24.5 | 24.5 | 25.5 | 25.5 |

### Observation:

#231—exhibited the desired gelation temperature (30-36 C). however during stress study lowering of gelation temperature is observed.
#236 and #243—Elevation in gelation temperature is observed during stress study
#235 and #265—initial and stress study samples showed lower gelation temperature (<30 C)

### Optimization of Lipoid S 100 Concentration

The Lipoid S 100 concentration in the formulation was optimized in the formulation by varying the Lipoid S 100 concentration. The compositions are presented as below:

TABLE 8
Composition for formulation with Lipoid S 100 (Optimization)

Batch no

| | POC-0718/ | POC-0718/ | POC-0718/ |
|---|---|---|---|
| | 231/P | 263/P | 264/p |
| Poloxamer 407 | 12 | 12 | 12 |
| Transcutol | 10 | 10 | 10 |
| Lipoid S 100 | 2 | 1 | 1.5 |
| Purified water QS | 93.33 | 93.33 | 93.33 |
| to | | | |
| Tgel (deg C.) | 33.5 | 40 | 33 |

### Manufacturing Process:

### Batch no POC-0718/231/P

### Batch no POC-0718/263/P

### Batch no POC-0718/264/P

### Stress Study:

Formulation with 1.5% of Lipoid S 100 shows gelation, whereas the formulation with 1% of Lipoid S100 didn't gel when heated up to 50 C.

Stress study (60 C) was conducted on the formulation (#264). Gelation temperature was monitored during the study. The results are presented as below:

TABLE 9
Stress study results for optimized Lipoid
S 100 containing formulations

| | | 60° C., 7 | 60° C., | 60° C., | 60° C., |
|---|---|---|---|---|---|
| Batch no | Initial | days | 14 days | 21 days | 28 days |
| POC- | 33.5 | 25.5 | 24.5 | 25.5 | 27.5 |
| 0718/264/P | | | | | |

### Observation:

At initial time point desired gelation temperature (30-36 C) is observed. However during stress study lowering of gelation temperature is observed. Based on the stress study, a minimum of 1.5% Lipoid S 100 is required to show thermogelling behavior.

### Stability Batches:

Formulation with 2% Lipoid S 100 has been considered as the lead formula. Reproducible batches with batch size (3-4 Kg) were manufactured and stability was monitored. These batches were packed into different packaging material MoC to understand its impact on the physical properties.

The compositions of the batches are provided as below:

TABLE 10
Composition for stability batches:

| Batch no | POC-0718/278/P | POC-0718/291/P | POC-0718/338/P | POC-0718/361/P |
|---|---|---|---|---|
| Poloxamer 407 | 12 | 12 | 12 | 12 |
| Transcutol | 10 | 10 | 10 | 10 |
| Lipoid S 100 | 2 | 2 | 2 | 2 |
| Purified water QS to | 93.33 | 93.33 | 93.33 | 93.33 |
| Source of Mesalamine | Cambrex | MSN | Cambrex | Cambrex |
| Grade of Mesalamine | SH | Micronized | S | S |
| MoC of packaging | EVOH coated | EVOH coated | HDPE | EVOH coated |
| material | HDPE bottle | HDPE bottle | | HDPE bottle |

TABLE 11
Stability data for POC-0718/278/P

| | | | | | | RS | |
|---|---|---|---|---|---|---|---|
| | | | Complex viscosity | | | | Total |
| | | | (mPas) | | | Single | impu- |
| Condition | Tg | pH | 25.2 | 37.2 | Assay | max | rities |
| Initial | 32.5 | 4.55 | 373 | 2,61,000 | 130.5 | 0.12 | 0.12 |
| 1 M, | 30.5 | 4.65 | 2,540 | 1,50,000 | 122.6 | 0.05 | 0.13 |
| 25/60 | | | | | | | |
| 3 M, | 31.5 | 4.57 | 5,590 | 89,300 | 118.5 | 0.13 | 0.23 |
| 25/60 | | | | | | | |
| 6 M, | 29.5 | 4.64 | 337 | 5,97,000 | NA | NA | NA |
| 25/60 | | | | | | | |
| 14 days | 31.5 | 4.74 | 5,130 | 2,54,000 | 35.3 | 0.11 | 0.25 |
| 40/75 | | | | | | | |
| 1 M, | 28.5 | 4.66 | 6,460 | 9,94,000 | 118.7 | 0.04 | 0.16 |
| 40/75 | | | | | | | |
| 3 M, | 27.5 | 4.6 | 12,900 | 82,600 | 120.2 | 0.15 | 0.33 |
| 40/75 | | | | | | | |
| 6 M, | 29.5 | 4.6 | 1020 | 8,16,000 | NA | NA | NA |
| 40/75 | | | | | | | |

TABLE 12
Stability data for POC-0718/291/P

| | | | | | | RS | |
|---|---|---|---|---|---|---|---|
| | | | Complex viscosity | | | | Total |
| | | | (mPas) | | | Single | impu- |
| Condition | Tg | pH | 25.2 | 37.2 | Assay | max | rities |
| Initial | 33.5 | 4.42 | 6,130 | 6,79,000 | 101.6 | NA | NA |
| 2 M, | 31.5 | 4.48 | 2,050 | 99,200 | 91.2 | 0.14 | 0.21 |
| 25/60 | | | | | | | |
| 3 M, | 31-32 | 4.53 | 1,940 | 6,55,000 | 97.5 | 0.08 | 0.17 |
| 25/60 | | | | | | | |
| 6 M, | 30-31 | 4.37 | 716 | 4,72,000 | | 0.07 | 0.19 |
| 25/60 | | | | | | | |
| 2 M, | 29.5 | 4.5 | 2,690 | 5,22,000 | 99.8 | 0.1 | 0.28 |
| 40/75 | | | | | | | |
| 3 M, | 29-30 | 4.54 | 3,550 | 8,36,000 | 90.1 | 0.09 | 0.4 |
| 40/75 | | | | | | | |
| 6 M, | 29-30 | 4.38 | 1,410 | 9,38,000 | | 0.07 | 0.26 |
| 40/75 | | | | | | | |

TABLE 13
Stability data for POC-0718/338/P

| | | | | | | RS | |
|---|---|---|---|---|---|---|---|
| | | | Complex viscosity | | | | Total |
| | | | (mPas) | | | Single | impu- |
| Condition | Tg | pH | 25.2 | 37.2 | Assay | max | rities |
| Initial | 33-34 | 4.62 | 481 | 4,74,000 | 100.5 | 0.01 | 0.03 |
| 1 M, | 31-32 | 4.57 | 4,050 | 4,50,000 | 103.7 | 0.03 | 0.03 |
| 25/60 | | | | | | | |
| 2 M, | 31-32 | 4.55 | 389 | 5,68,000 | | 0.02 | 0.05 |
| 25/60 | | | | | | | |
| 3 M, | 32-33 | 4.53 | 5520 | 2,04,000 | | 0.02 | 0.04 |
| 25/60 | | | | | | | |
| 6 M, | 31-32 | 4.52 | 1530 | 4,00,000 | | 0.01 | 0.01 |
| 25/60 | | | | | | | |
| 9 M, | 31-32 | 4.50 | 3120 | 2,07,000 | | | |
| 25/60 | | | | | | | |
| 1 M, | 29-30 | 4.54 | 3,910 | 8,04,000 | 103.8 | 0.03 | 0.03 |
| 40/75 | | | | | | | |
| 2 M, | 30-31 | 4.5 | 485 | 6,71,000 | | 0.03 | 0.06 |
| 40/75 | | | | | | | |
| 3 M, | 28-29 | 4.49 | 9690 | 9,05,000 | | 0.02 | 0.06 |
| 40/75 | | | | | | | |
| 6 M, | 31-32 | 4.25 | 5,700 | 8,50,000 | | 0.01 | 0.01 |
| 40/75 | | | | | | | |

TABLE 14
Stability data for POC-0718/361/P

| | | | | | | RS | |
|---|---|---|---|---|---|---|---|
| | | | Complex viscosity | | | | Total |
| | | | (mPas) | | | Single | impu- |
| Condition | Tg | pH | 25.2 | 37.2 | Assay | max | rities |
| Initial | 33-34 | 4.59 | 322 | 4,81,000 | 100 | 0.08 | 0.17 |
| 1 M, | 29-30 | 4.65 | 656 | 7,03,000 | 99.6 | 0.08 | 0.14 |
| 25/60 | | | | | | | |
| 2 M, | 29-30 | 4.65 | 257 | 9,90,000 | | 0.03 | 0.07 |
| 25/60 | | | | | | | |
| 3 M, | 30-31 | 4.62 | 262 | 8,44,000 | | 0.02 | 0.04 |
| 25/60 | | | | | | | |
| 6 M, | 29-30 | 4.77 | | | | | |
| 25/60 | | | | | | | |
| 9 M, | 30.0 | 4.73 | 7,400 | 3,58,000 | | | |
| 25/60 | | | | | | | |
| 1 M, | 29-30 | 4.63 | 1,570 | 2,88,000 | 103.1 | 0.08 | 0.21 |
| 40/75 | | | | | | | |
| 2 M, | 30-31 | 4.63 | 468 | 11,60,000 | | 0.01 | 0.03 |
| 40/75 | | | | | | | |
| 3 M, | 28-29 | 4.61 | 1,700 | 11,80,000 | | 0.03 | 0.08 |
| 40/75 | | | | | | | |
| 6 M, | 29-30 | 4.74 | | | | | |
| 40/75 | | | | | | | |

### Observation:

The formulations irrespective of the source of API have the consistent in gelation temperature (diluent and reconstituted). No significant increase in the impurity profile and other test parameters were observed.

During the formulation development, it has been observed that additives like PVP and Poloxamer 188 elevate the gelation temperature whereas BHT lowers the gelation temperature. Further experimentations were carried out to screen the additive(s).

### Impact of BHT in Lipoid S 100 Formulation

In few formulations, BHT reduces the gelation temperature. This may further lower the concentration of Poloxamer 407 in the formulation. Experiments with different concentration of BHT with lowering of Poloxamer were executed to understand its impact on the gelation temperature. The compositions are presented as below:

TABLE 15
Composition for formulation with BHT

| Batch no | POC-0718/302/P | POC-0718/303/P | POC-0718/304/P |
|---|---|---|---|
| Poloxamer | 11 | 11 | 10 |
| 407 | | | |
| Transcutol | 12 | 15 | 15 |
| BHT | 0.5 | 0.2 | 0.5 |
| Lipoid S 100 | 2 | 2 | 2 |
| Purified water | 93.33 | 93.33 | 93.33 |
| QS to | | | |
| Tgel (deg C.) | | | |

### Impact of Poloxamer 188 in Lipoid S 100 Formulation

Based on compatibility study (Ref: POC-0718/269/P, POC-0718/270/P and POC-0718/272/P) Poloxamer 188 elevates the gelation temperature. The current lead formulation shows the gelation temperature close to 30 C, in order to achieve the desired gelation temperature of 32-36 C formulations with different concentration of Poloxamer 188 were executed. The compositions are presented as below:

TABLE 16
Composition for formulation with Poloxamer 188

| Batch no | POC-0718/294/P | POC-0718/296/P | POC-0718/485/P | POC-0718/486/P | POC-0718/487/P |
|---|---|---|---|---|---|
| Poloxamer 407 | 12 | 12 | 12.5 | 12.5 | 12.5 |
| Poloxamer 188 | 0.1 | 0.2 | 0.1 | 0.2 | 0.3 |
| Transcutol | 10 | 10 | 10 | 10 | 10 |
| Lipoid S 100 | 2 | 1 | 2 | 2 | 2 |
| Purified water QS | 93.33 | 93.33 | 93.33 | 93.33 | 93.33 |
| to | | | | | |
| Tgel (deg C.) | | | 29-30 | 31-32 | 33-34 |

### Observation:

With addition of Poloxamer 188, elevation of gelation temperature is observed. To understand the long term storage, stability was monitored for 2 prototypes with 0.1 and 0.300 Poloxamer 188.

Stability Batches with 0.1% Poloxamer 188:

Formulations with 0.10 Poloxmer 188 were manufactured. The stability was monitored with different grades and sources of Mesalamine. The compositions and the stability details are as below:

TABLE 17
Composition for Stability batches with 0.1% Poloxamer 188

| Batch no | POC-0718/320/P | POC-0718/331/P | POC-0718/445/P | POC-0718/533/P |
|---|---|---|---|---|
| Poloxamer 407 | 12 | 12 | 12 | 12 |
| Poloxamer 188 | 0.1 | 0.1 | 0.1 | 0.1 |
| Transcutol | 10 | 10 | 10 | 10 |
| Lipoid S 100 | 2 | 2 | 2 | 2 |
| Purified water QS | 93.33 | 93.33 | 93.33 | 93.33 |
| to | | | | |
| Source of | NA | Cambrex | Cambrex | MSN |
| Mesalamine | | | | |

TABLE 18
Stability data for POC-0718/320/P

| Condition | Tg (Diluent) | pH (Diluent) |
|---|---|---|
| Initial | 33-34 | 7.29 |
| 40/75, 1 M | 29-30 | 6.74 |
| 40/75, 2 M | 29-30 | 6.80 |
| 40/75, 3 M | 31-32 | 6.64 |
| 40/75, 6 M | 30-31 | 6.26 |
| 25/60, 1 M | 32-33 | 6.68 |
| 25/60, 2 M | 32-33 | 6.90 |
| 25/60, 3 M | 32-33 | 6.82 |
| 25/60, 6 M | 32-33 | 6.69 |

TABLE 19
Stability data for POC-0718/331/P

| | | | | | Complex viscosity | | | RS | |
|---|---|---|---|---|---|---|---|---|---|
| | Tg | | pH | | (mPas)-Reconstituted | | Assay of | Single | Total |
| Condition | Diluent | Reconstituted | Diluent | Reconstituted | 25.2° C. | 37.2° C. | Mesalamine | max | impurity |
| Initial | 35-36 | 35-36 | 7.17 | 4.62 | 282 | 1,47,000 | 95.9 | NA | NA |
| 40/75, 1 M | 31-32 | 31-32 | 6.77 | 4.62 | 3,890 | 12,70,000 | 101.4 | 0.09 | 0.28 |
| 40/75, 2 M | 30-31 | 30-31 | 6.75 | 4.62 | 5,870 | 11,40,000 | 100.7 | 0.09 | 0.22 |
| 40/75, 3 M | 28-29 | 28-29 | 6.68 | 4.65 | 5,740 | 2,53,000 | | 0.02 | 0.04 |
| 40/75, 6 M | 30-31 | 30-31 | 6.16 | 4.62 | 4,870 | 10,90,000 | | | |
| 25/60, 1 M | 33-34 | 33-34 | 6.97 | 4.59 | 2310 | 9,77,000 | 102.4 | 0.08 | 0.2 |
| 25/60, 2 M | 33-34 | 33-34 | 6.9 | 4.63 | 3,870 | 9,38,000 | 102 | 0.08 | 0.19 |
| 25/60, 3 M | 32-33 | 32-33 | 6.92 | 4.63 | 3,220 | 5,38,000 | | 0.01 | 0.04 |
| 25/60, 6 M | 33-34 | 33-34 | 6.80 | 4.70 | 2,170 | 8,84,000 | | | |
| 25/60, 9 M | 33.5-34.5 | 33.5-34.5 | 6.03 | 4.75 | 1260 | 1,57,000 | | | |

TABLE 20
Stability data for POC-0718/445/P

| | | | | | Complex viscosity | | | RS | |
|---|---|---|---|---|---|---|---|---|---|
| | Tg | | pH | | (mPas)-Reconstituted | | Assay of | Single | Total |
| Condition | Diluent | Reconstituted | Diluent | Reconstituted | 25.2° C. | 37.2° C. | Mesalamine | max | impurity |
| Initial | 33-34 | 32-33 | 7.26 | 4.58 | — | — | | 0.01 | 0.01 |
| 40/75, 1 M | — | 30-31 | — | 4.58 | 3,860 | 9,06,000 | | 0.04 | 0.04 |
| 40/75, 2 M | — | 30-31 | — | 4.52 | 2,530 | 8,28,000 | | 0.03 | 0.03 |
| 40/75, 3 M | 32-33 | 32-33 | 6.94 | 4.72 | | | | | |
| 40/75, 6 M | | 33-34 | — | 4.65 | | | | | |
| 25/60, 1 M | — | 32-33 | — | 4.58 | 3,300 | 8,13,000 | | 0.01 | 0.01 |
| 25/60, 2 M | — | 33-34 | — | 4.53 | 3,590 | 6,86,000 | | 0.06 | 0.08 |
| 25/60, 3 M | 33-34 | 33-34 | 6.97 | 4.73 | | | | | |
| 25/60, 6 M | | 33-34 | — | 4.46 | | | | | |

TABLE 21
Stability data for POC-0718/533/P

| | | | | | Complex | | | | |
|---|---|---|---|---|---|---|---|---|---|
| | | | | | viscosity | | | | |
| | | | | | (mPas)- | | | RS | |
| | Tg | | pH | | Reconstituted | | Assay of | Single | Total |
| Condition | Diluent | Reconstituted | Diluent | Reconstituted | 25.2° C. | 37.2° C. | Mesalamine | max | impurity |

| | | | | |
|---|---|---|---|---|
| Initial | 33-34 | 34-35 | 7.25 | 4.55 |
| 25/60, | 30-31 | 31-32 | 7.08 | 4.61 |
| 1 M | | | | |
| 40/75, 1 M | 30-31 | 31-32 | 7.08 | 4.66 |

Stability Batches with 0.3% Poloxamer 188:

Formulations with 0.3% Poloxmer 188 were manufactured. The stability was monitored with different grades and sources of Mesalamine. The compositions and the stability details are as below:

TABLE 22
Composition for Stability batches with 0.3% Poloxamer 188

| Ingredients | POC-0718/498/P | POC-0718/534/P |
|---|---|---|
| Lipoid S 100 | 2 | 2 |
| Poloxamer 407 | 12.5 | 12.5 |
| Poloxamer 188 | 0.3 | 0.3 |
| Transcutol P | 10 | 10 |
| Purified water | QS to 93.33 | QS to 93.33 |
| Source of | Cambrex | MSN |
| Mesalamine | | |
| Grade of | S | Micronized |
| mesalamine | | |

TABLE 24
Stability data for POC-0718/498/P and POC-0718/534/P

| Batch no | Condition | Tg | pH |
|---|---|---|---|
| POC- | Initial | 32-33 | 4.76 |
| 0718/498/P | 1 M, 25/60 | 29-30 | 4.76 |
| | 2 M, 25/60 | 29-30 | 4.88 |
| | 1 M, 40/75 | 28-29 | 4.81 |
| | 2 M, 40/75 | 23-24 | 4.96 |
| POC- | Initial | 32-33 | 4.60 |
| 0718/534/P | | | |

### Impact of Polyvinyl Pyrrolidone (PVP) in Lipoid S 100 Formulation

### POC-0718/253-rn

Based on compatibility study (Ref: POC-0718/253-m) PVP K-30 elevates the gelation temperature. Experiments with different concentration of PVP (Kollidon 30) were executed to understand its impact on the gelation temperature. The compositions are presented as below:

TABLE 25
composition for formulation with PVP

| | POC- | POC- | POC- | POC- | POC- | POC- | POC- |
|---|---|---|---|---|---|---|---|
| Batch no | 0718/402/P | 0718/404/P | 0718/415/P | 0718/407/P | 0718/432/P | 0718/444/P | 0718/483/P |
| Poloxamer | 12 | 12 | 13 | 12 | 12 | 12 | 12 |
| 407 | | | | | | | |
| PVP | 0.5 | 0.1 | 0.1 | 0.05 | 0.05 | 0.05 | 0.05 |
| (Kollidon | | | | | | | |
| 30) | | | | | | | |
| Transcutol | 10 | 10 | 10 | 10 | 10 | 10 | 10 |
| Lipoid S | 2 | 2 | 2 | 2 | 2 | 2 | 2 |
| 100 | | | | | | | |
| Purified | 93.33 | 93.33 | 93.33 | 93.33 | 93.33 | 93.33 | 93.33 |
| water | | | | | | | |
| QS to | | | | | | | |
| Tgel | >40 | >40 | 27-28 | 33-34 | 33-34 | 33-34 | 33-34 |
| (deg C.) | | | | | | | |

Observation: Among all experiments #432 and #444 showed desired gelation temperature. These formulations were subjected to ICH stability study to understand it's impact.

TABLE 26
Stability data for POC-0718/432/P

| | Tg | pH |
|---|---|---|
| Condition | (Reconstituted) | (Reconstituted) |
| Initial | 34-35 | 4.55 |
| 1 M, | 33-34 | 4.58 |
| 25° C./60% RH | | |
| 2 M, | 32-33 | 4.49 |
| 25° C./60% RH | | |
| 3 M, | 33-34 | 4.62 |
| 25° C./60% RH | | |
| 6 M, | 31-32 | 4.63 |
| 25° C./60% RH | | |
| 1 M, | 30-31 | 4.58 |
| 40° C./75% RH | | |
| 2 M, | 30-31 | 4.45 |
| 40° C./75% RH | | |
| 3 M, | 33-34 | 4.57 |
| 40° C./75% RH | | |
| 6 M, | > 40 | |
| 40° C./75% RH | | |

TABLE 27
Stability data for POC-0718/444/P

| | Tg | pH |
|---|---|---|
| Condition | (Reconstituted) | (Reconstituted) |
| Initial | 33-34 | 4.57 |
| 1 M, | 33-34 | 4.57 |
| 25° C./60% RH | | |
| 2 M, | 33-34 | 4.53 |
| 25° C./60% RH | | |
| 3 M, | 32-33 | 4.71 |
| 25° C./60% RH | | |
| 6 M, | 33-34 | 4.63 |
| 25° C./60% RH | | |
| 1 M, | 31-32 | 4.58 |
| 40° C./75% RH | | |
| 2 M, | 30-31 | 4.52 |
| 40° C./75% RH | | |
| 3 M, | 31-32 | 4.68 |
| 40° C./75% RH | | |
| 6 M, | > 40 | 4.27 |
| 40° C./75% RH | | |

Elevation in gelation temperature is observed during stability study. Significant changes in the gelation temperature is observed in formulations stored at accelerated condition. 6M 40/75 sample didn't gel even at 40 C. Considering the instability at accelerated condition, the strategy was discontinued.

### Impact of Edetate Disodium in Lipoid S 100 Formulation

Experiments with addition of Edetate disodium were executed to understand its impact on the gelation temperature. The compositions are presented as below:

TABLE 28
composition for formulation with Edetate disodium

| | POC- | POC- |
|---|---|---|
| Ingredients | 0718/456/P | 0718/468/P |
| Edetate sodium | 0.1 | 0.1 |
| Transcutol | 10 | 10 |
| Lipoid S 100 | 2.0 | 2.0 |
| Poloxamer 407 | 12.0 | 12.0 |
| Purified water | QS to 93.33 | QS to 93.33 |
| Tg (° C.) | 31-32 | 33-34 |
| pH | 6.00 | 6.06 |

### Impact of Phosphate Buffer in Lipoid S 100 Formulation

pH for the reconstituted formulation is lower than the diluent. Mesalamine undergoes degradation at lower pH. To control pH for the diluent and reconstituted formulation, phosphate buffer of pH 7.4 and 8.0 were evaluated. The compositions for the formulations are presented as below:

TABLE 29
composition for formulation with phosphate buffer

| Ingredients | POC-0718/474/P | POC-0718/476/P |
|---|---|---|
| Transcutol | 10 | 10 |
| Lipolid S 100 | 2 | 2 |
| Poloxamer 407 | 12 | 12 |
| Phosphate buffer | QS to 93.33 (pH 7.4) | QS to 93.33 (pH 8.0) |
| Tg (Diluent) | 25-26 | 25-26 |
| pH(Diluent) | 7.84 | 8.34 |
| Tg (Reconstituted) | 32-33 | >40 |
| pH(Reconstituted) | 6.28 | 6.32 |

### Observation:

Irrespective of different buffers, lowering of pH after reconstitution is observed. Also increase in gelation temperature of the reconstituted formulation is observed. Further the strategy was discontinued

### Reconstitution Study

The lead prototype (#278/P) was reconstitution with Mesalamine (MSN). The gelation temperature was monitored periodically up to 24 hours.

TABLE 30
Gelation temperature for reconstituted
formulation over a period of time

| Time | Gelation temperature |
|---|---|
| (hr) | (C.) |
| 0 | 32 |
| 1 | 32 |
| 2 | 33 |
| 3 | 33 |
| 6 | 33 |
| 8 | 33 |
| 15 | 36 |
| 18 | 36 |
| 24 | 38 |

Observation: No significant changes in gelation temperature were observed up to 8 hours post reconstitution. Thereafter increase in gelation temperature is observed.

### Formulation with Phospholipon 90 G

Phospholipon 90 G is an unsaturated phospholipid with lower transition temperature. Multiple experiments were executed to understand the impact of formulation on the gelation temperature. Composition for the prototype formulations are presented below:

TABLE 31
Composition for formulation with Phospholipon 90 G

| | POC- | POC- |
|---|---|---|
| | 0718/250/P | 0718/251/P |
| Edetate disodium | 0.1 | 0.1 |
| Phospholipon 90 | 1 | 2 |
| G | | |
| Poloxamer 407 | 12.5 | 12.5 |
| Polycarbophil | 0.25 | 0.25 |
| Sodium chloride | 0.5 | 0.5 |
| TPGS | 0.2 | 0.2 |
| Transcutol | 10 | 10 |
| Tris | 0.15 | 0.15 |
| Water | q.s. to 93.33 | q.s. to 93.33 |
| Gelation | Gelled at RT | Gelled at RT |
| temperature | | |

TABLE 32
Composition for formulation with Phospholipon 90 G

| Batch no | POC-0718/201/P | POC-0718/255/P | POC-0718/256/P | POC-0718/261/P | POC-0718/262/P |
|---|---|---|---|---|---|
| Poloxamer 407 | 12 | 12.5 | 13 | 12.5 | 12.5 |
| Edetate disodium | | 0.1 | 0.1 | 0.1 | 0.1 |
| Transcutol | 10 | 10 | 10 | 10 | 10 |
| Phospholipon 90 | 1 | 1 | 1 | 0.5 | 0.75 |
| G | | | | | |
| Purified water QS | 93.33 | 93.33 | 93.33 | 93.33 | 93.33 |
| to | | | | | |
| Tgel (deg C.) | 40 | 28 | 27 | 33 | 31 |

### Stress Study:

In order to understand the impact of heat and time on the gelation temperature, all these formulations were kept at 60 C for 4 weeks. The gelation temperature was monitored every week. The results are presented as below:

TABLE 33
Stress study results

| Batch no | Initial | 7 days | 14 days | 21 days | 28 days |
|---|---|---|---|---|---|
| POC-0718/261/P | 33.5 | 33.5 | 40 | 40 | 40 |
| POC-0718/262/P | 31.5 | 31.5 | 31.5 | 32.5 | 32.5 |

Formulation with 0.5% Phospholipon 90 G:

TABLE 34
Composition for formulation with 0.5% Phospholipon 90 G

| Ingredients | POC-0718/279/P |
|---|---|
| Edetate sodium | 0.1 |
| Phospholipon 90G | 0.5 |
| Poloxamer 407 | 12.5 |
| Transcutol P | 10.0 |
| Purified water | QS to 93.33 |

TABLE 35
Stress study results

| | Diluent | | Cambrex | | MSN | |
|---|---|---|---|---|---|---|
| Condition | Tg | pH | Tg | pH | Tg | pH |
| Initial | 31.5 | 4.57 | | | | |
| 1 w 60° C. | 35.5 | | 35.5 | 4.64 | 35.5 | 4.54 |
| 2 w 60° C. | >40 | — | >40 | 4.73 | >40 | 4.63 |
| 3 w 60° C. | >40 | — | >40 | 4.74 | >40 | 4.62 |
| 4 w 60° C. | >40 | | >40 | 4.67 | >40 | 4.57 |

TABLE 36
Stability data for POC-0718/279/P

| | | | Complex viscosity | | | RS | |
|---|---|---|---|---|---|---|---|
| | Tg | | (mPas) | | | | Total |
| Condition | 10 min | pH | 25.2 | 37.2 | Assay | Single max | impurity |
| Initial | 31.5 | 4.57 | 313 | 3,83,000 | 99 | 0.22 | 0.12 |
| Initial | 31.5 | 4.65 | 520 | 7,30,000 | 78.9 | 0.16 | 0 |
| 14 days 40/75 | 34.5 | 4.72 | 2,130 | 1,60,000 | 101.1 | 0.09 | 0.24 |
| 1 M, 40/75 | >40 | 4.66 | 1,420 | 1,33,000 | 129.4 | 0.04 | 0.13 |
| 1 M, 25/60 | 33.5 | 4.66 | 3,200 | 1,09,000 | 104.9 | 0.07 | 0.17 |
| 3 M, 40/75 | >40 | 4.64 | 17,700 | 36,800 | 122.8 | 0.21 | 0.39 |
| 3 M, 25/60 | >40 | 4.66 | 1,260 | 40,500 | 114.7 | 0.18 | 0.33 |

Stability with 0.75% Phospholipon 90 G:

Multiple reproducible batches of #262/P have been manufactured and stability was monitored with different sources/grades of mesalamine to understand its impact. The compositions are provided as below:

TABLE 37
Composition for formulation with 0.75% Phospholipon 90 G

| Ingredients | POC-0718/280/P | POC-0718/293/P | POC-0718/339/P | POC-0718/362/P |
|---|---|---|---|---|
| Edetate sodium | 0.1 | 0.1 | 0.1 | 0.1 |
| Transcutol | 10 | 10 | 10 | 10 |
| Phospholipon 90 G | 0.75 | 0.75 | 0.75 | 0.75 |
| Poloxamer 407 | 12.5 | 12.5 | 12.5 | 12.5 |
| Purified water | QS to 93.33 | QS to 93.33 | QS to 93.33 | QS to 93.33 |
| Source of Mesalamine | Cambrex | MSN | Cambrex | Cambrex |
| Grade of mesalamine | SH | Micronized | S | S |
| MoC of primary | EVOH coated | EVOH coated | HDPE bottle | EVOH coated |
| packaging material | HDPE bottle | HDPE bottle | | HDPE bottle |
| Tg (° C.) | 32-33 | 31-32 | 32-33 | 32-33 |
| pH | 6.03 | 6.11 | 6.07 | 6.08 |

TABLE 38
Stability data for POC-0718/280/P

| | | | Complex | | | RS | |
|---|---|---|---|---|---|---|---|
| | | | viscosity (mPas) | | | Single | Total |
| Condition | Tg | pH | 25.2 | 37.2 | Assay | max | impurities |
| Initial | 31.5 | 4.65 | 520 | 7,30,000 | 85.3 | 0.25 | 0.47 |
| 1 M, 40/75 | 30.5 | 4.65 | 2,680 | 1,98,000 | 110.9 | 0.07 | 0.27 |
| 3 M, 40/75 | 33 | 4.66 | 7,650 | 2,39,000 | 112.3 | 0.48 | 0.49 |
| 6 M, 40/75 | 35.5 | 4.67 | 269 | 1,39,000 | NA | NA | NA |
| 1 M, 25/60 | 30.5 | 4.66 | 2,910 | 1,46,000 | 103.9 | 0.05 | 0.18 |
| 3 M, 25/60 | 30.5 | 4.64 | 3,580 | 3,85,000 | 118.5 | 0.48 | 0.56 |
| 6 M, 25/60 | 34.5 | 4.66 | 267 | 1,62,000 | NA | NA | NA |

TABLE 39
Stability data for POC-0718/293/P

| | | | Complex | | | RS | |
|---|---|---|---|---|---|---|---|
| | | | viscosity (mPas) | | | Single | Total |
| Condition | Tg | pH | 25.2 | 37.2 | Assay | max | impurities |
| Initial | 31.5 | 4.45 | 6,570 | 9,61,000 | 100 | NA | NA |
| 2 M, 40/75 | 34.5 | 4.58 | 1,250 | 2,06,000 | 94.9 | 0.3 | 0.39 |
| 3 M, 40/75 | 34.5 | 4.54 | 1050 | 3,66,000 | 97.2 | 0.09 | 0.24 |
| 6 M, 40/75 | >40 | 4.47 | NA | NA | NA | NA | NA |
| 2 M, 25/60 | 33.5 | 4.58 | 5,880 | 3,03,000 | 92.3 | 0.28 | 0.39 |
| 3 M, 25/60 | 33.5 | 4.55 | 1630 | 5,86,000 | 93.6 | 0.09 | 0.27 |
| 6 M, 25/60 | >40 | 4.47 | NA | NA | NA | NA | NA |

TABLE 40
Stability data for POC-0718/339/P

| | | | Complex | | | RS | |
|---|---|---|---|---|---|---|---|
| | | | viscosity (mPas) | | | Single | Total |
| Condition | Tg | pH | 25.2 | 37.2 | Assay | max | impurities |
| Initial | 31.5 | 4.59 | 353 | 2,50,000 | 98.1 | 0.04 | 0.07 |
| 1 M, 40/75 | 32.5 | 4.58 | 2,090 | 3,68,000 | 101.9 | 0.04 | 0.12 |
| 2 M, 40/75 | 31.5 | 4.57 | 621 | 7,15,000 | UA | 0.01 | 0.06 |
| 3 M, 40/75 | 33-34 | 4.54 | 2,560 | 3,53,000 | | 0.01 | 0.02 |
| 6 M, 40/75 | >40 | 4.54 | — | — | | | |
| 1 M, 25/60 | 31.5 | 4.58 | 3,200 | 3,72,000 | 103 | 0.04 | 0.13 |
| 2 M, 25/60 | 32.5 | 4.58 | 723 | 7,55,000 | UA | 0.01 | 0.02 |
| 3 M, 25/60 | 33-34 | 4.55 | 5,660 | 2,23,000 | | 0.01 | 0.04 |
| 6 M, 25/60 | 31-32 | 4.59 | 6260 | 247000 | | | |

TABLE 41
Stability data for POC-0718/362/P

| | | | Complex | | | RS | |
|---|---|---|---|---|---|---|---|
| | | | viscosity (mPas) | | | Single | Total |
| Condition | Tg | pH | 25.2 | 37.2 | Assay | max | impurities |
| Initial | 32.5 | 4.58 | 1,270 | 5,66,000 | 101.1 | 0.08 | 0.20 |
| 1 M, 40/75 | 31.5 | 4.64 | 4890 | 1,55,000 | 104.4 | 0.05 | 0.13 |
| 2 M, 40/75 | 31.5 | 4.67 | 254 | 1,75,000 | UA | 0.01 | 0.03 |
| 3 M, 40/75 | 34-35 | 4.61 | 457 | 2,90,000 | | 0.01 | 0.03 |
| 6 M, 40/75 | >40 | | | | | | |
| 1 M, 25/60 | 31.5 | 4.63 | 780 | 1,15,000 | 101.2 | 0.06 | 0.15 |
| 2 M, 25/60 | 32.5 | 4.65 | 265 | 2,10,000 | UA | 0.01 | 0.01 |
| 3 M, 25/60 | 33-34 | 4.61 | 301 | 3,17,000 | | 0.01 | 0.04 |
| 6 M, 25/60 | >40 | | | | | | |

### Observation:

Non uniformity of mesalamine is observed with Cambrex SH grade. The selected grade of the mesalamine is agglomerated and difficulties in getting uniform dispersion is observed during reconstitution.
Formulation is found to be stable in HJDPE bottles
Instability is observed in both the batches packed in EVOH coated HJDPE bottles

### Impact of Poloxamer 188 in Lipoid S 100 Formulation

Based on compatibility study (Ref: POC-0718/269/P, POC-0718/270/P and POC-0718/272/P) Poloxamer 188 elevates the gelation temperature. The current lead formulation shows the gelation temperature close to 30 C, in order to achieve the desired gelation temperature of 32-36 C formulations with different concentration of Poloxamer 188 were executed. The compositions are presented as below:

TABLE 42
Composition for Phospholipon 90
G formulation with Poloxamer 188

| Batch no | POC-0718/295/P | POC-0718/296/P |
|---|---|---|
| Poloxamer 407 | 12.5 | 12.5 |
| Poloxamer 188 | 0.1 | 0.1 |
| Edetate disodium | 0.1 | 0.1 |
| Transcutol | 10 | 10 |
| Phospholipon 90 | 0.5 | 0.75 |
| G | | |
| Purified water | 93.33 | 93.33 |
| QS to | | |
| Tgel (deg C.) | 33 | 31 |

### Impact of BHT in Phospholipon 90 G Formulation

In few formulations, BHT reduces the gelation temperature. This may further lower the concentration of Poloxamer 407 in the formulation. Experiments with different concentration of BHT with lowering of Poloxamer were executed to understand its impact on the gelation temperature. The compositions are presented as below:

TABLE 43
Composition for Phospholipon 90 G formulation with BHT

| | POC- | POC- | POC- | POC- | POC- | POC- | POC- | POC- | POC- | POC- | POC- |
|---|---|---|---|---|---|---|---|---|---|---|---|
| | 0718/ | 0718/ | 0718/ | 0718/ | 0718/ | 0718/ | 0718/ | 0718/ | 0718/ | 0718/ | 0718/ |
| Batch no | 340/P | 341/P | 342/P | 343/P | 344/P | 345/P | 346/P | 347/P | 348/P | 355/P | 356/P |
| Poloxamer | 11 | 10 | 11 | 10.5 | 10.5 | 10.5 | 11 | 11 | 11 | 11 | 11 |
| 407 | | | | | | | | | | | |
| Edetate | 0.1 | 0.1 | 0.1 | 0.1 | 0.1 | 0.1 | 0.1 | 0.1 | 0.1 | 0.1 | 0.1 |
| disodium | | | | | | | | | | | |
| Transcutol | 10 | 10 | 10 | 10 | 10 | 10 | 10 | 10 | 10 | 10 | 10 |
| BHT | 0.5 | 0.5 | 0.5 | 0.4 | 0.5 | 0.5 | 0.4 | 0.5 | 0.5 | 0.5 | 0.5 |
| Phospholipon | 0.75 | 0.75 | 0.5 | 0.75 | 0.5 | 0.75 | 0.5 | 0.4 | 0.3 | 0.25 | 0.2 |
| 90 G | | | | | | | | | | | |
| Purified | 93.33 | 93.33 | 93.33 | 93.33 | 93.33 | 93.33 | 93.33 | 93.33 | 93.33 | 93.33 | 93.33 |
| water QS | | | | | | | | | | | |
| to | | | | | | | | | | | |
| Tgel (deg | 25-26 | >40 | 30-31 | >40 | >40 | >40 | >40 | 31-32 | 33-34 | 33.5-34.5 | >40 |
| C.) | | | | | | | | | | | |

### Impact of Edetate Disodium in Phospholipon 90 G Formulation

Experiments with addition of Edetate disodium were executed to understand its impact on the gelation temperature. The compositions are presented as below:

TABLE 44
composition for formulation without Edetate disodium

| Batch no | POC-0718/473/P |
|---|---|
| Poloxamer 407 | 12.5 |
| Transcutol | 10 |
| Phospholipon 90 G | 0.75 |
| Purified water QS | 93.33 |
| to | |
| Tgel (deg C.) | 32.0 C. |

TABLE 45
Stability data for POC-0718/473/P

| | | | Complex Viscosity(m · Pa · s) | |
|---|---|---|---|---|
| | Diluent | Reconstituted | At | At |
| | Tg | Tg | 25.2 C. | 37.2 C. |
| Initial | 32-33 | 32-33 | — | — |
| 1 M, 25/60 | 31-32 | 31-32 | — | — |
| 2 M, 25/60 | 31-32 | 31-32 | 451 | 6,05,000 |
| 3 M, 25/60 | 31-32 | 31-32 | 2800 | 10,40,000 |
| 6 M, 25/60 | 32-33 | 32-33 | | |
| 9 M, 25/60 | 29-30 | 29-30 | | |
| 12 M, 25/60 | 32-33 | 32-33 | | |
| 1 M, 40/75 | 31-32 | 31-32 | | |
| 2 M, 40/75 | 31-32 | 31-32 | 763 | 7,16,000 |
| 3 M, 40/75 | 31-32 | 31-32 | 2890 | 7,68,000 |
| 6 M, 40/75 | 32-33 | 32-33 | | |

### Conclusion

Phospholipon 90G formulation without Edetate disodium shows no changes in gelation temperature during stability up to 12 months.

### Impact of Phosphate Buffer in Phospholipon 90 G Formulation

pH for the reconstituted formulation is lower than the diluent. Mesalamine undergoes degradation at lower pH. To control pH for the diluent and reconstituted formulation, phosphate buffer of pH 7.4 and 8.0 were evaluated. The compositions for the formulations are presented as below:

TABLE 46
composition for formulation with phosphate buffer

| Ingredients | POC-0718/ 458/P | POC-0718/ 475/P | POC-0718/ 477/P |
|---|---|---|---|
| Edetate sodium | 0.1 | — | — |
| Transcutol | 10 | 10 | 10 |
| PG 90 | 0.75 | 0.75 | 0.75 |
| Poloxamer 407 | 12.5 | 12.5 | 12.5 |
| Phosphate buffer | QS to 93.33 | QS to 93.33 | QS to 93.33 |
| | (pH 7.0) | (pH 7.4) | (pH 8.0) |
| Tg (diluent) | 33-34 | 25-26 | 25-26 |
| pH (diluent) | 7.31 | 7.81 | 8.35 |
| Tg (Reconstituted) | 34-35 | 33-34 | >40 |
| pH (Reconstituted) | 5.91 | 6.24 | 6.37 |

### Drug Suspended in Transcutol

To avoid spillage during reconstitution (API to the diluent), Mesalamine was dispersed in transcutol containing Lipid and the Poloxamer phase in the other container. The composition of the formulation is provided as below:

### Liquid Compartment Trial 2 Stability

Compartment-1: EDTA & Poloxamer P 407 in purified water
Compartment-2: PG 90G, 5-ASA slurry in Transcutol

TABLE 47
composition for 2 compartment formulation

| | POC-0718/455 |
|---|---|
| Ingredients | % w/w |
| 5-ASA | 6.67 |
| Edetate sodium | 0.1 |
| Phospholipon 90 G | 0.75 |
| Poloxamer 407 | 13 |
| Transcutol P | 10 |
| Purified water | QS |
| Tg | 32-33 |

TABLE 48
Stability data for POC-0718/455/P

Reconstituted

| POC-0718/455 | Description | Reconstituted Tg |
|---|---|---|
| Initial | Light pink | 32-33 |
| 1 M, 25/60 | Light pink | 32-33 |
| 1 M, 40/75 | Light pink | 33-34 |
| 2 M, 25/60 | Light pink | 31-32 |
| 2 M, 40/75 | Light brown | 31-32 |

### Reconstitution Study

The lead prototype (#279/P and #280/P) was reconstitution with Mesalamine (Source—MSN).

The gelation temperature was monitored periodically up to 24 hours.

TABLE 49
Gelation temperature of reconstituted formulation over time

| | POC-0718/279/P + | POC-0718/280/P + |
|---|---|---|
| Time (hours) | MSN | MSN |
| 0 | 34 | 31 |
| 1 | 33 | 31 |
| 2 | 34 | 31 |
| 3 | 34 | 32 |
| 6 | 34 | 32 |
| 8 | 34 | 33 |
| 15 | 40 | 35 |
| 18 | 40 | 35 |
| 24 | 40 | 36 |

Observation: No significant changes in gelation temperature were observed up to 8 hours post reconstitution. Thereafter increase in gelation temperature is observed.

### Formulation with Lipoid S 100 and Phospholipon 90 G

During stress study of formulation with Phospholipon 90 G shows elevating in gelation temperature whereas with Lipoid S100 lowering in the gelation temperature is observed. In order to have the consistent gelation temperature during stability, mixture of both the lipids were evaluated. The compositions are presented as below:

TABLE 50
composition for formulation containing Lipoid S 100 and Phospholipon 90 G

| | POC- | POC- | POC- | POC- | POC- | POC- |
|---|---|---|---|---|---|---|
| Batch no | 0718/417/P | 0718/418/P | 0718/419/P | 0718/423/P | 0718/424/P | 0718/425/P |
| Poloxamer 407 | 12.25 | 12.25 | 12.25 | 12 | 12 | 12 |
| Edetate | 0.1 | 0.1 | 0.1 | 0.1 | 0.1 | 0.1 |
| disodium | | | | | | |
| Transcutol | 10 | 10 | 10 | 10 | 10 | 10 |
| Phospholipon 90 | 0.375 | 0.1875 | 0.5625 | 0.375 | 0.1875 | 0.5625 |
| G | | | | | | |
| Lipoid S 100 | 1 | 1.5 | 0.5 | 1 | 1.5 | 0.5 |
| Purified water | 93.33 | 93.33 | 93.33 | 93.33 | 93.33 | 93.33 |
| QS to | | | | | | |
| Tgel (deg C.) | 32-33 | 30-31 | 32-33 | >40 | 34.5-35.5 | >40 |

### Observation:

#417, #419 and #424 showed desired gelation temperature. Stability study was initiated for the selected prototypes.

### Stability Study

The compositions of the stability batches are presented as below:

TABLE 51
composition for stability batches containing Lipoid S 100 and Phospholipon 90 G

| Batch no | POC-0718/429/P | POC-0718/430/P | POC-0718/431/P |
|---|---|---|---|
| Poloxamer 407 | 12.25 | 12.25 | 12 |
| Edetate disodium | 0.1 | 0.1 | 0.1 |
| Transcutol | 10 | 10 | 10 |
| Phospholipon 90 | 0.375 | 0.5625 | 0.1875 |
| G | | | |
| Lipoid S 100 | 1 | 0.5 | 1.5 |
| Purified water QS | 93.33 | 93.33 | 93.33 |
| to | | | |
| Tgel (deg C.) | 32-33 | 33-34 | 34-35 |

TABLE 52
Stability data for POC-0718/430/P

| Batch no. | Condition | Tg | pH |
|---|---|---|---|
| POC-0718/430/P | Initial | 33.5 | |
| | 1 M, | 33.5 | |
| | 25° C./60% RH | | |
| | 2 M, | 35.5 | |
| | 25° C./60% RH | | |
| | 3 M, | >40 | |
| | 25° C./60% RH | | |
| | 1 M, | 33.5 | |
| | 40° C./75% RH | | |
| | 2 M, | 35.5 | |
| | 40° C./75% RH | | |
| | 3 M, | >40 | |
| | 40° C./75% RH | | |
| POC-0718/429/P | Initial | 32.5 | |
| | 1 M, | 32.5 | |
| | 25° C./60% RH | | |
| | 2 M, | 33.5 | |
| | 25° C./60% RH | | |
| | 3 M, | 34.0 | |
| | 25° C./60% RH | | |
| | 1 M, | 30.5 | |
| | 40° C./75% RH | | |
| | 2 M, | 33.5 | |
| | 40° C./75% RH | | |
| | 3 M, | 33.5 | |
| | 40° C./75% RH | | |
| POC-0718/431/P | Initial | 34.5 | |
| | 1 M, | 33.5 | |
| | 25° C./60% RH | | |
| | 2 M, | 34.5 | |
| | 25° C./60% RH | | |
| | 3 M, | >40 | |
| | 25° C./60% RH | | |
| | 1 M, | 33.5 | |
| | 40° C./75% RH | | |
| | 2 M, | 34.5 | >40 |
| | 40° C./75% RH | | |
| | 3 M, | | |
| | 40° C./75% RH | | |

TABLE 53
Mixing of the two containers:

| Order of API | | Type of | HDPE bottle | | |
|---|---|---|---|---|---|
| addition | Shaking time | shaking | capacity | Tg (° C.) | Appearance |
| API followed by | 15 sec | Gentle | 120 ml | 33-34 (10 min | API lumps at the |
| vehicle | | | | till 4 h) | bottom |
| API followed by | 30 sec | Vigorous | 120 ml | 33-34 (10 min | Lump free |
| vehicle | | | | till 4 h) | suspension |
| API followed by | 15 sec | Vigorous | 120 ml | 33-34 (10 min | Lump free |
| vehicle | | | | till 4 h) | suspension |
| Vehicle followed | 10 sec | Vigorous | 120 ml | 33-34 (10 min | API adhered to the |
| by API | | | | till 4 h) | sidewalls of the |
| | | | | | bottle |
| Vehicle followed | shaking-1min | Vigorous | 120 ml | 33-34 (10 min | Lump free |
| by API | 10 sec | | | till 4 h) | suspension |
| | hold-10 sec | | | | |
| | shaking | | | | |
| API followed by | 10 sec | Vigorous | 120 ml | 33-34 (10 min | Lump free |
| vehicle | shaking-1min | | | till 4 h) | suspension |
| | hold-10 sec | | | | |
| | shaking | | | | |
| API followed by | 10 sec | Vigorous | 60 ml | 33-34 (10 min | API lumps were |
| vehicle | shaking-1min | | | till 4 h) | observed at the |
| | hold-10 sec | | | | bottom |
| | shaking | | | | |
| Vehicle followed | shaking-1min | Vigorous | 60 ml | 33-34 (10 min | API lumps were |
| by API | 10 sec | | | till 4 h) | observed at the |
| | hold-10 sec | | | | bottom |
| | shaking | | | | |

### INT-CL-001 Phase 1 Comparative Pharmacokinetic (PK) Study Designed to Demonstrate Advantages Over Standard of Care Enema ROWASA® in Q012022

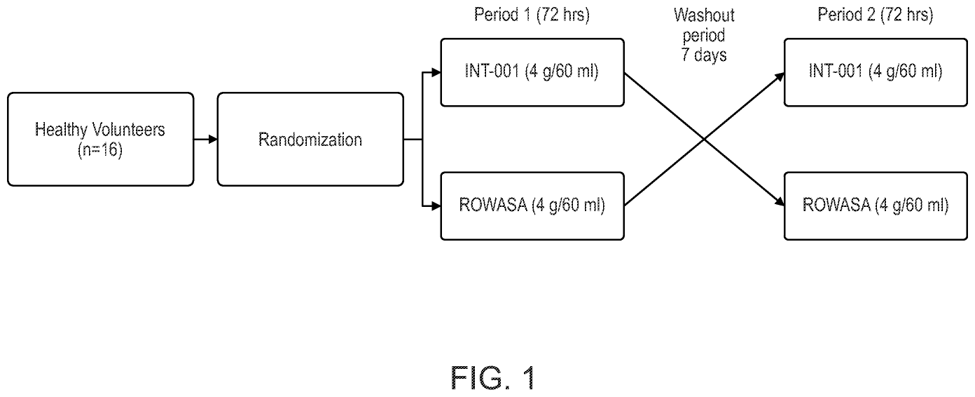

Endpoints for the study were to demonstrate better retention and improved absorption of 5-ASA vs. standard of care; the addition of stool PK to provide estimates of mucosal tissue concentration (correlated with efficacy. The layout of the study is illustrated in FIG. 1.

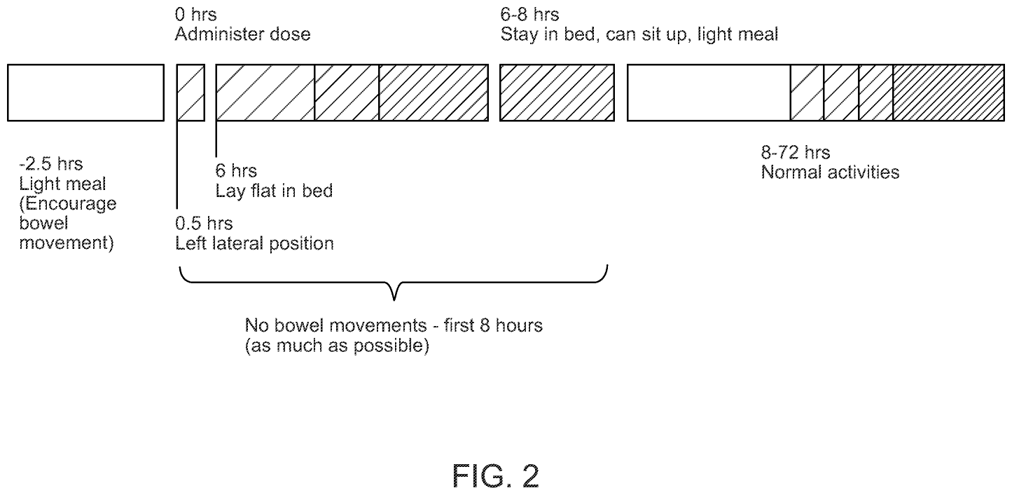

FIG. 2 shows a study period dosing and PK sampling timeline. PK sampling intervals included PK blood draws every hour for first 12 hours, then at hours 18, 24, 36, 48, 60, and 72 (±10 mins); urine pooled from hours 0 to 4, 4 to 8, 8 to 12, 12 to 24, 24 to 48, 48 to 72; and stool pooled from 0 to 8, 8 to 24, 24 to 48, 48 to 72. Vital signs were taken 30 min before PK sample. Washout was 7 days in-house.

Table 54 shows the dosing overview and details for the INT-CL-001 Phase 1 PK study:

| | Period 1 | Period 2 |
|---|---|---|
| No. of subject planned | 16 + | 16 |
| | additional | |
| No. of subjects Dosed | 16 | 16 |
| Subject withdrawal | NA | 01 (Adverse Event) |
| with reason | | |
| Adverse event details | NA | Blood in stool passed, Subject |
| | | 08, 14:51 on 16 May 2022 |
| Number of subjects | 16 | 15 |
| completing Period | | |

Table 55 shows single dose PK in healthy volunteers with select FDA-approved 5-ASA products:

| | 5-ASA | | | | | | | |
|---|---|---|---|---|---|---|---|---|
| | | Tmax | | | n-acetyl-5-ASA | | | |
| | Cmax | h, | AUCinf | | Cmax | | AUCinf | |
| | ng/ml, | mean | ng · h/ml, | t½ h, | ng/ml, | Tmax | ng · h/ml, | t½ h, |
| | mean ± | (% CV | mean ± | mean ± | mean ± | h, | mean ± | mean ± |
| | SD | OR | SD | SD | SD | mean | SD | SD |
| Product | (% CV) | range) | (% CV) | (% CV) | (% CV) | (range) | (% CV) | (% CV) |

INT-CL-001 Results

| | | | | | | | | |
|---|---|---|---|---|---|---|---|---|
| ROWASA | 1,100 | 6.88 | 11,300 | 2.73 | 2,650 | 8.88 | 31,800 | 3.56 |
| rectal | (70.2) | (61.9) | (80.6) | (35.5) | (65.4) | (42.5) | (79.4) | (1.25) |
| suspension | | | | | | | | |
| enema, 4 g/ | | | | | | | | |
| 60 ml | | | | | | | | |
| INT-001 | 883 | 6 | 11,926 | 5.24 | 2,303 | 7.45 | 40,298 | 4.15 |
| rectal | (71.8) | (70.7) | (75.5) | (90.9)* | (74.3) | (49.7) | (82.3) | (37.6) |
| suspension | | | | | | | | |
| enema, 4 g/ | | | | | | | | |
| 60 ml | | | | | | | | |

Reference Products

| | | | | | | | | |
|---|---|---|---|---|---|---|---|---|
| ROWASA | 1,500.3 | 6.8 | 26,862.9 | 10.3 | — | — | — | — |
| rectal | (51.9) | | (79.7) | | | | | |
| suspension | | | | | | | | |
| enema, 4 g/ | | | | | | | | |
| 60 ml | | | | | | | | |
| CANASA | 192.75 | 2.30 | 1,697.7 | 3.96 | 475.90 | 4.10 | 4,487.4 | 3.48 |
| rectal | (53.33) | (60.90) | (96.3) | (119.81) | (56.65) | (43.90) | (95.29) | (99.31) |
| suppository, | | | | | | | | |
| 500 mg | | | | | | | | |
| LIALDA | 4,385 ± | 8.04 | 48,141 ± | 6.28 ± | 5,535 ± | 9.0 | 109,498 ± | 12.6 ± |
| oral tablet, | 3,033 | (6.0-32.1) | 25,627 | 5.31 | 2,596 | (6.0-36.1) | 44,838 | 11.3 |
| delayed | | | | | | | | |
| release, 4.8 | | | | | | | | |
| g, (4 × 1.2 | | | | | | | | |
| g) tablet, | | | | | | | | |
| fed | | | | | | | | |
| APRISO | 2,130 ± | 4 | 13,570 ± | 9.2 ± | 2,780 ± | 4 | 50,620 ± | 12.4 ± |
| oral | 1,100 | (2-16) | 5,440 | 7.1 | 850 | (4-12) | 23,060 | 10.8 |
| capsule, | | | | (59.3) | (30.5) | | | |
| extended | | | | | | | | |
| release, 1.5 | | | | | | | | |
| g, (4 × | | | | | | | | |
| 0.375 g), | | | | | | | | |
| fasting | | | | | | | | |
| ASACOL | 5,000 ± | (10-16) | 20,000 ± | 12.6 ± | 4,600 ± | (10-16) | 25,000 ± | 23.6 ± |
| oral tablet, | 4,000 | | 14,000 | 10.9 | 2,500 | | 11,000 | 11.2 |
| delayed | | | | | | | | |
| release, 4.8 | | | | | | | | |
| g, (2 × 800 | | | | | | | | |
| mg, 3x | | | | | | | | |
| daily), | | | | | | | | |
| fasting | | | | | | | | |

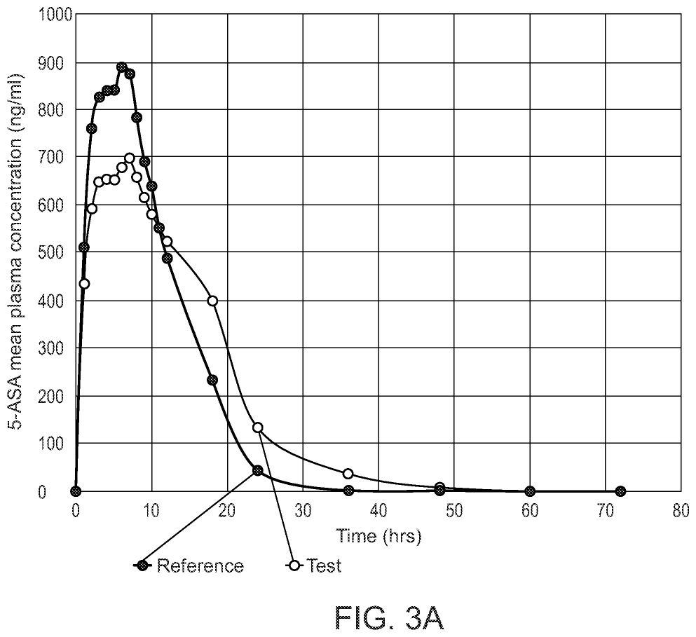

FIG. 3 shows plasma PK for the test compound (INT-001) vs. the reference compound (ROWASA). FIG. 3A shows mesalamine while FIG. 3B shows n-acetyl-mesalamine.

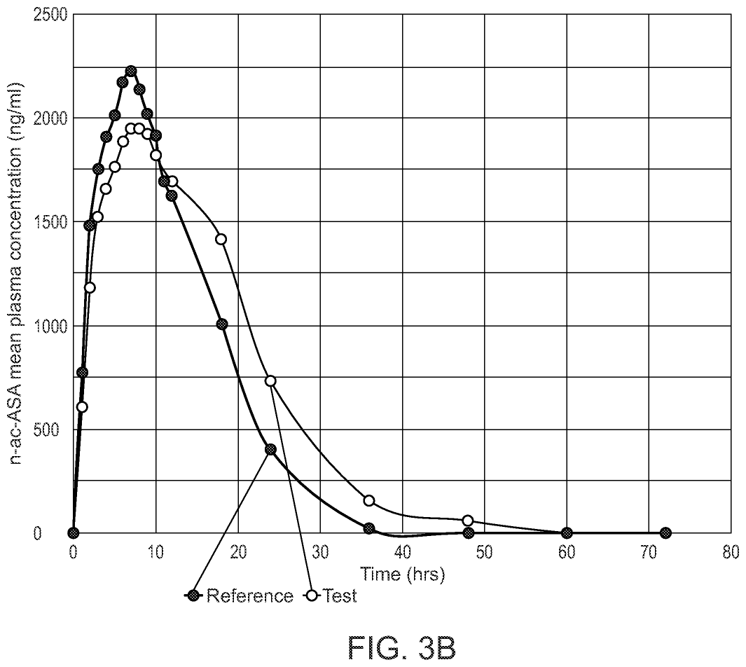

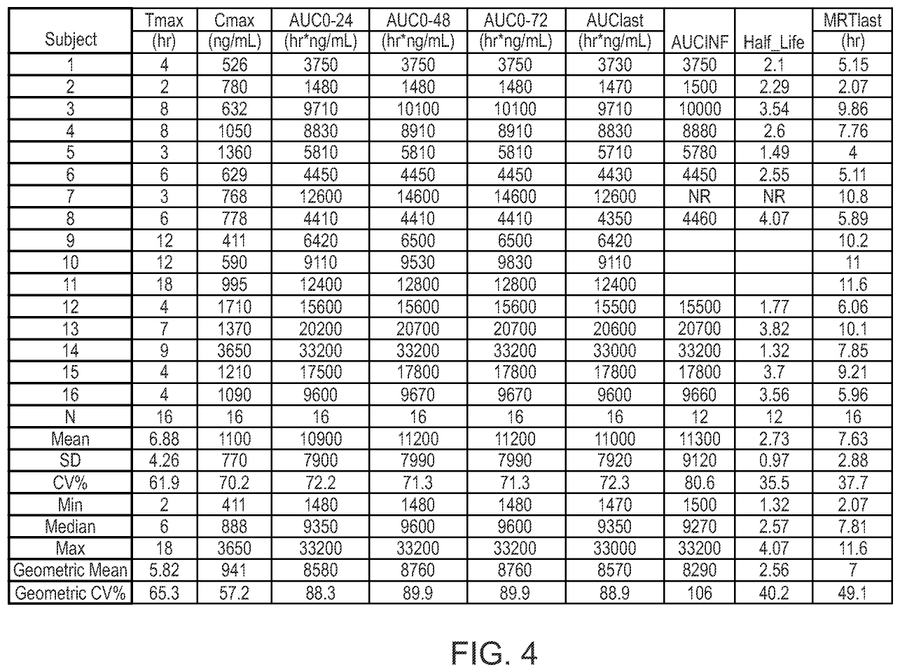

FIG. 4 shows the mesalamine reference data while FIG. 5 shows data for the test compound.

Table 56 shows the bioequivalence calculation for the test and reference:

Test/Reference (%)

90% CI

| Parameter | Ratio | Lower | Upper |
|---|---|---|---|
| Cₘₐₓ (ng/mL) | 77.5 | 56.1 | 107 |
| AUC₀₋₂₄ (ng-h/mL) | 97.2 | 67.7 | 140 |
| AUC₀₋₄₈ (ng-h/mL) | 103 | 69.9 | 152 |
| AUC₀₋₇₂ (ng-h/mL) | 103 | 70.0 | 153 |
| AUCₗₐₛₜ (ng-h/mL) | 97.6 | 64.3 | 148 |

Table 57 shows time to first stool vs. MRT:

| | Test | | | Reference | | |
|---|---|---|---|---|---|---|
| | Time to | MRTlast | MRTlast | Time to | MRTlast | MRTlast |
| | First Stool | (5-ASA) | (n-ac-5- | First Stool | (5-ASA) | (n-ac-5- |
| | (hrs) | (hrs) | ASA) (hrs) | (hrs) | (hrs) | ASA) (hrs) |
| mean | 10.7 | 9.8 | 11.9 | 11.9 | 7.6 | 9.6 |
| SD | 7.5 | 4.2 | 4.8 | 7.9 | 2.9 | 3.2 |
| median | 7.89 | 8.70 | 10.90 | 8.78 | 7.81 | 9.59 |
| max | 22.83 | 16.60 | 19.50 | 24.32 | 11.60 | 14.70 |
| min | 2.32 | 2.69 | 3.02 | 1.98 | 2.07 | 3.43 |

Table 58 shows subgroup analysis by time to first stool for 0-4 and 4-8 hours:

| | First Stool | | | | | | | |
|---|---|---|---|---|---|---|---|---|
| | 0-4 hours | | | | 4-8 hours | | | |
| | | Cmax | AUC72 | MRTlast | | Cmax | AUC72 | MRTlast |
| Parameter | n | (ng/ml) | (hr*ng/mL) | (hrs) | n | (ng/ml) | (hr*ng/mL) | (hrs) |
| Reference | 3 | 925.00 | 5782.67 | 5.31 | 5 | 713.74 | 6209.21 | 6.88 |
| Test | 3 | 541.37 | 7331.39 | 10.25 | 4 | 722.07 | 6382.45 | 7.62 |

Table 59 shows subgroup analysis by time to first stool for 8-12 and 12+ hours:

| | First Stool | | | | | | | |
|---|---|---|---|---|---|---|---|---|
| | 8-12 hours | | | | 12+ hours | | | |
| | | Cmax | AUC72 | MRTlast | | Cmax | AUC72 | MRTlast |
| Parameter | n | (ng/ml) | (hr*ng/mL) | (hrs) | n | (ng/ml) | (hr*ng/mL) | (hrs) |
| Reference | 1 | 1085.92 | 9670.38 | 5.96 | 7 | 1446.77 | 17324.22 | 9.41 |
| Test | 2 | 942.48 | 9570.00 | 7.36 | 6 | 1245.30 | 19717.87 | 12.91 |

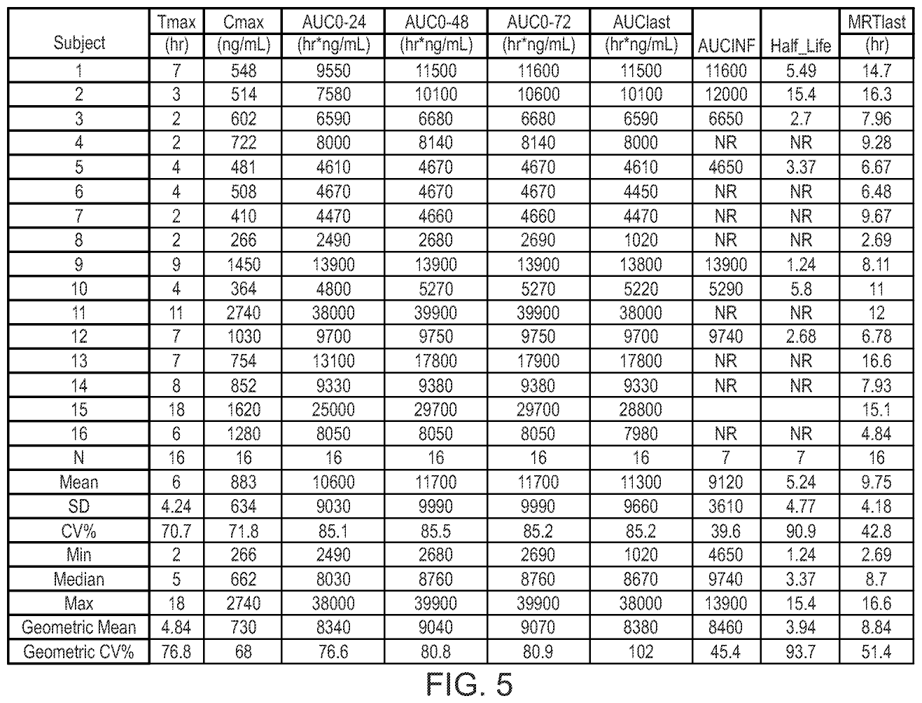

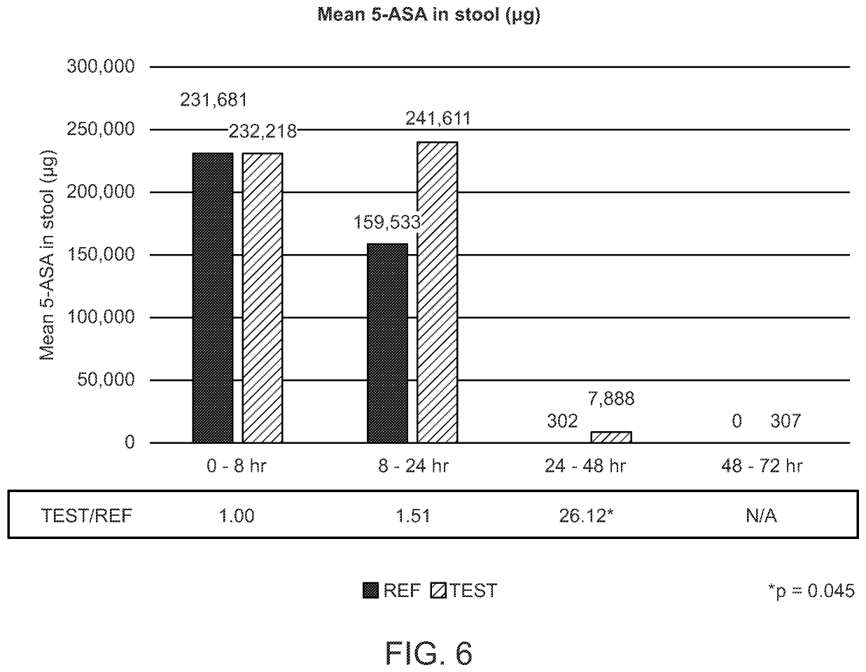

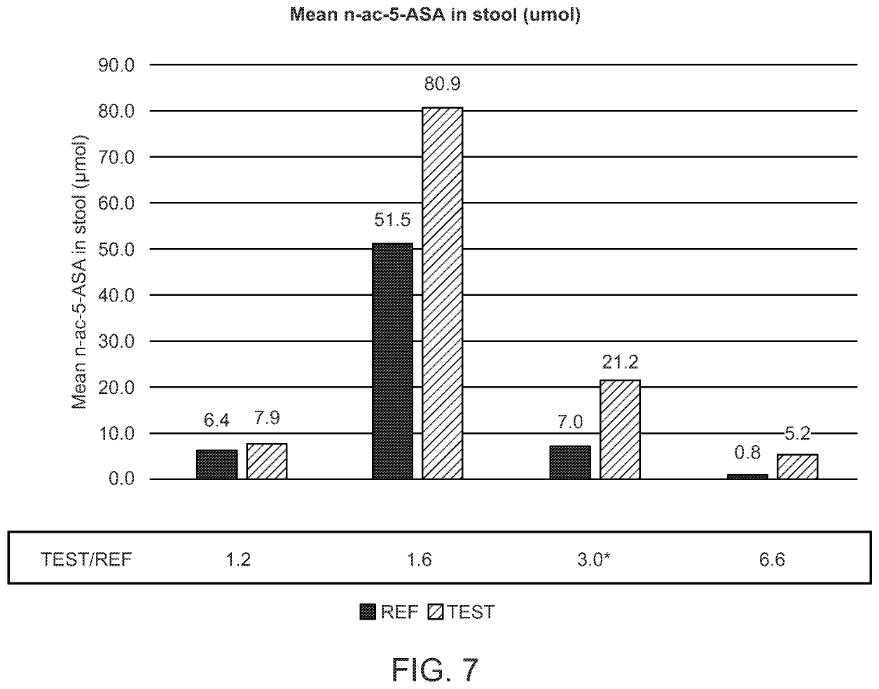

FIG. 6 shows mean 5-ASA in stool and FIG. 7 shows mean n-ac-5-ASA in stool for the test and reference compounds. The reference compound appears on the left while the test compound appears on the right of each coupled bar in the charts. FIG. 8 shows time to first stool after administration of the reference and the test compounds for six example test subjects plotted on plasma PK curves (y-axis is plasma 5-ASA concentration in ng/ml and x-axis is hours after administration). For all but one (subject 9) the first stool occurred more quickly with the reference than the test compound. In these examples, the plasma concentration of mesalamine drops to zero after a bowel movement for subjects after receiving the reference compound. However, for subjects receiving the test compound, mesalamine levels remained elevated for 18-48 hours after the bowel movement, indicating likely that the fractions of the gel are retained in the colon despite the subject having had a bowel movement. For reference, prior 5-ASA studies include for ROWASA mesalamine enema in 1987 (4 g/60 mL), CANASA mesalamine suppository in 2001 (500 mg), and UCERIS budesonide rectal foam in 2014 (2 mg rectal foam 505b2). For additional reference, see Dilger K et al. A clinical trial on absorption and N-acetylation of oral and rectal mesalazine. Eur J Clin Invest. 2007 July; 377:558-65; Aumais G et al. Pharmacokinetics and Pilot Efficacy of a Mesalazine Rectal Gel in Distal Ulcerative Colitis. Drugs R D 2005; 61:41-46; and Aumais G et al. Rectal tissue, plasma and urine concentrations of mesalazine after single and multiple administrations of 500 mg suppositories to healthy volunteers and ulcerative proctitis patients. Aliment Pharmacol Ther 2003; 17:93-97; the content of each of which is incorporated herein by reference in its entirety. Additional data reelavant to 5-ASA dose response can be found, for example, in Hanauer SB. Dose-ranging study of mesalamine PENTASA enemas in the treatment of acute ulcerative proctosigmoiditis: results of a multicentered placebo-controlled trial. The U.S. PENTASA Enema Study Group. Inflamm Bowel Dis. 1998 May; 42:79-83; Campieri M et al. Optimum dosage of 5-aminosalicylic acid as rectal enemas in patients with active ulcerative colitis. Gut 1991, 32, 929-931; Frieri G. Mucosal 5-aminoslicylic acid concentration inversely correlates with severity of colonic inflammation in patients with ulcerative colitis. Gut 2000; 47:410-4141; and Naganuma M. Measurement of colonic mucosal concentrations of 5-aminoslicylic acid is useful for estimating its therapeutic efficacy in distal ulcerative colitis: comparison of orally administered mesalamine and sulfasalazine. IBD 2001; 7(3):221-225; the content of each of which is incorporated herein by reference in its entirety.

### Formulation Studies

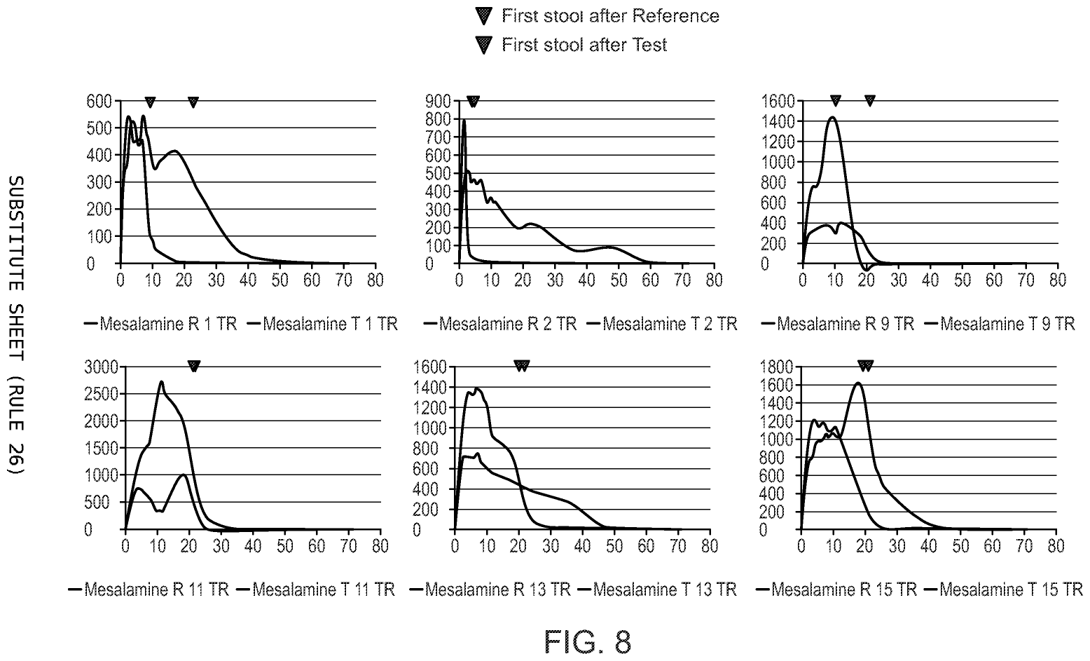

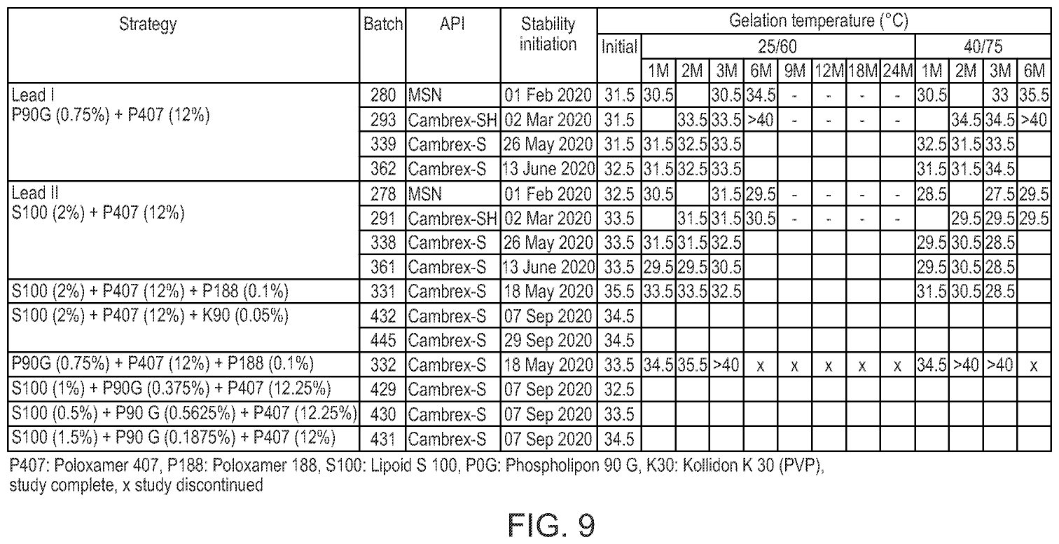

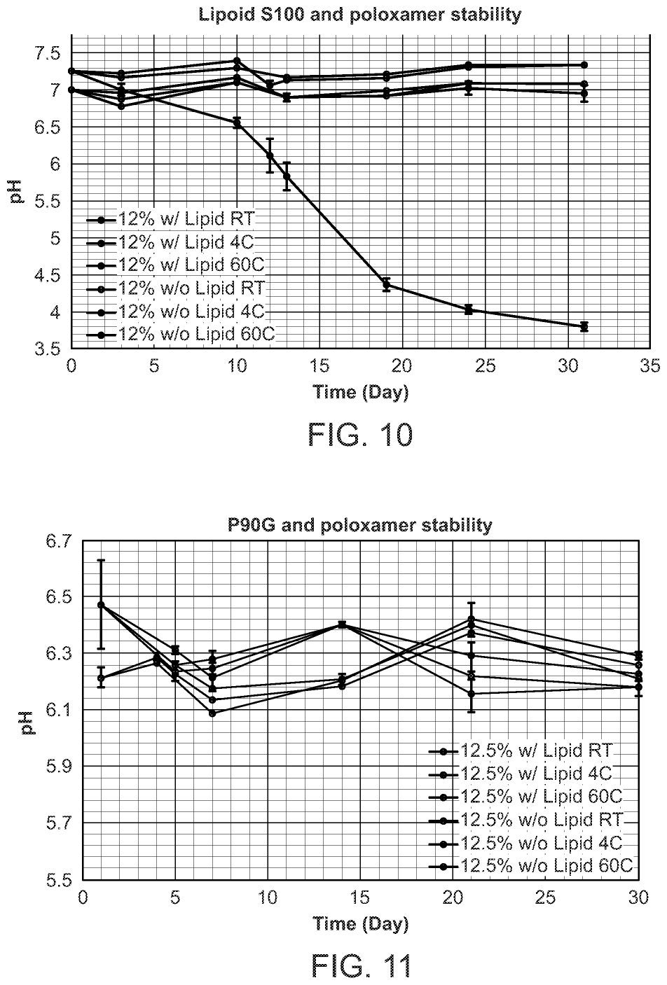

Stability data for various compound formulations is shown in FIG. 9. Formulation I (P90G) and II (S100) were stored with and without lipids at 4° C., RT and 60° C. PH was used as an indicator for detecting possible degradation. Formulation I (P90G) was found to perform better at 60° C. as compared to formulation II (S100). FIG. 10 shows poloxamer stability for lipoid S100 and FIG. 11 shows poloxamer stability for p90G.

### One-Part and Two-Part Formulations

Data for two exemplary non-lipid based one-part formulations is shown in Table 67:

| | POC-0718/109 | | POC-718/114 | |
|---|---|---|---|---|
| Ingredients | POC-718/147 | POC-718/137/P | POC-0718/148 | POC-718/136/P |
| Mesalamine | 6.67 | — | 6.67 | — |
| Edetate sodium | 0.1 | 0.1 | 0.1 | 0.1 |
| Poloxamer 407 | 15 | 15 | 14.0 | 14.0 |
| Polycarbophil | 0.25 | 0.25 | 0.25 | 0.25 |
| Nacl | 0.5 | 0.5 | 0.5 | 0.5 |
| Tris | 0.15 | 0.15 | 0.15 | 0.15 |
| Vitamin E TPGS | — | — | 0.2 | 0.2 |
| Transcutol | — | — | 10.0 | 10.0 |
| Purified water | q.s. to 100 | q.s. to 93.33 | q.s. to 100 | q.s. to 93.33 |
| pH | 4.78 | 5.01 | 4.91 | 5.16 |
| Gelation temp. (° C.) | 24-25 | 23.5 to 24.5 | 23.5 to 24.5 | 25-26 |
| Complex Viscosity at 37° C. | 9,05,000 | — | 6,95,000 | — |
| | Active | Placebo | Active | Placebo |

Stability data for the above one-part formulations is shown in Table 68:

| | | | | RS of Mesalamine | | |
|---|---|---|---|---|---|---|
| | | | Assay | @ | @ | |
| | | Complex | of | RRT | RRT | |
| Condition | Tg (° C.) | viscosity | Mesalamine | 2.3 | 3.1 | Total |

POC-0718/148 (Active)

| | | | | | | |
|---|---|---|---|---|---|---|
| Initial | 23.5 to 24.5 | 6,95,000 | 102.4 | 0.04 | ND | 0.24 |
| 25° C./60% RH, 1 M | 28-29 | 6,48,000 | 100.7 | 0.08 | ND | 0.82 |
| 40° C./75% RH, 1 M | 29-30 | 5,49,000 | 99.0 | 0.17 | 0.02 | 1.50 |

POC-718/136/P (Placebo)

| | | | | | | |
|---|---|---|---|---|---|---|
| Initial | 25-26 | 7,18,000 | NA | NA | NA | NA |
| 25° C./60% RH, 1 M | 27.5-28.5 | 6,79,000 | NA | NA | NA | NA |
| 40° C./75% RH, 1 M | 26.5-27.5 | 6,72,000 | NA | NA | NA | NA |

POC-0718/147 (Active)

| | | | | | | |
|---|---|---|---|---|---|---|
| Initial | 24-25 | 9,05,000 | 99.8 | 0.03 | ND | 0.28 |
| 25° C./60% RH, 1 M | 27.5-28.5 | 7,00,000 | 98.5 | 0.07 | 0.06 | 0.67 |
| 40° C./75% RH, 1 M | 28-29 | 6,95,000 | 98.4 | 0.14 | 0.26 | 1.09 |

POC-718/137/P (Placebo)

| | | | | | | |
|---|---|---|---|---|---|---|
| Initial | 23.5 to 24.5 | 11,00,000 | NA | NA | NA | NA |
| 25° C./60% RH, 1 M | 26-27 | 10,20,000 | NA | NA | NA | NA |
| 40° C./75% RH, 1 M | 26-27 | 9,49,000 | NA | NA | NA | NA |

Formulations were stored at 60° C. Related substances were determined using HPLC. Changes in gelation temperature were observed in the active compounds. However, no changes in gelation temperature were observed for the placebo. Higher impurities were observed during stability analyses. In this study, one-part formulations containing mesalamine were not stable. Mesalamine was found to turn brown within 3 days after preparation of one-part formulations because of oxidation in an aqueous solution.

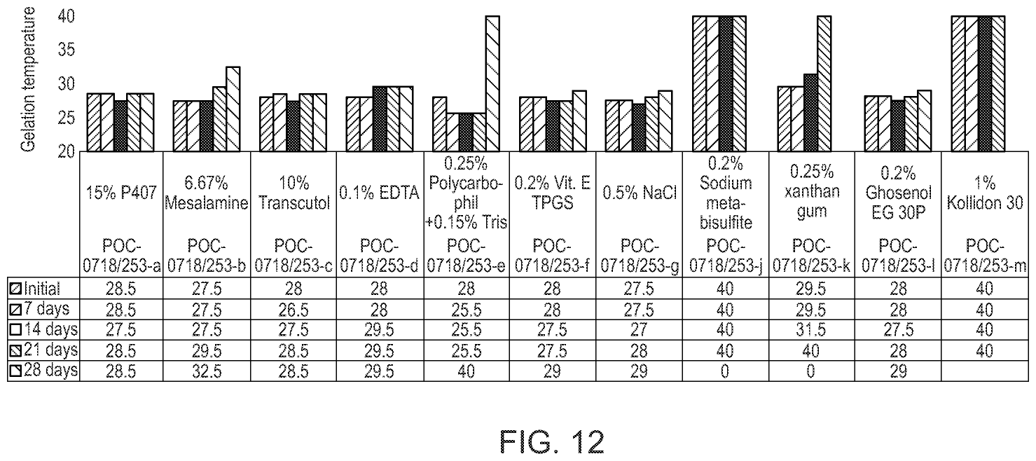

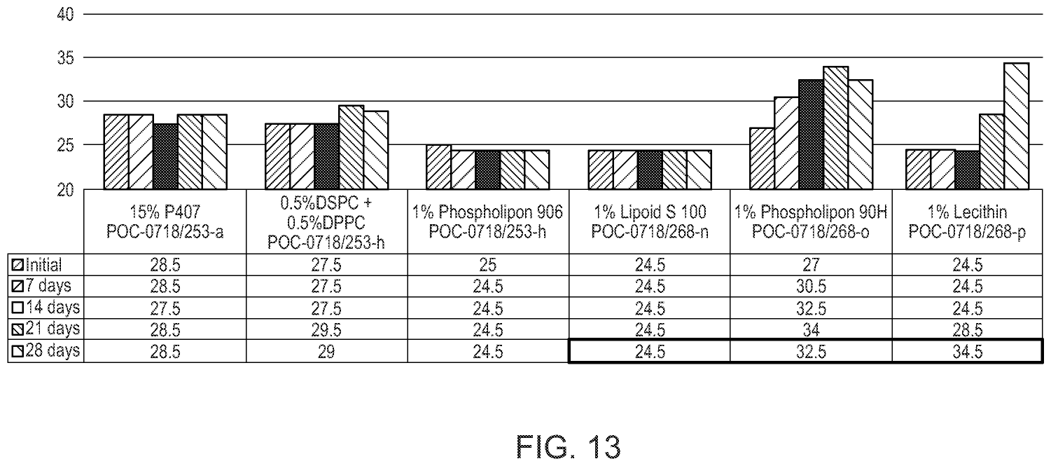

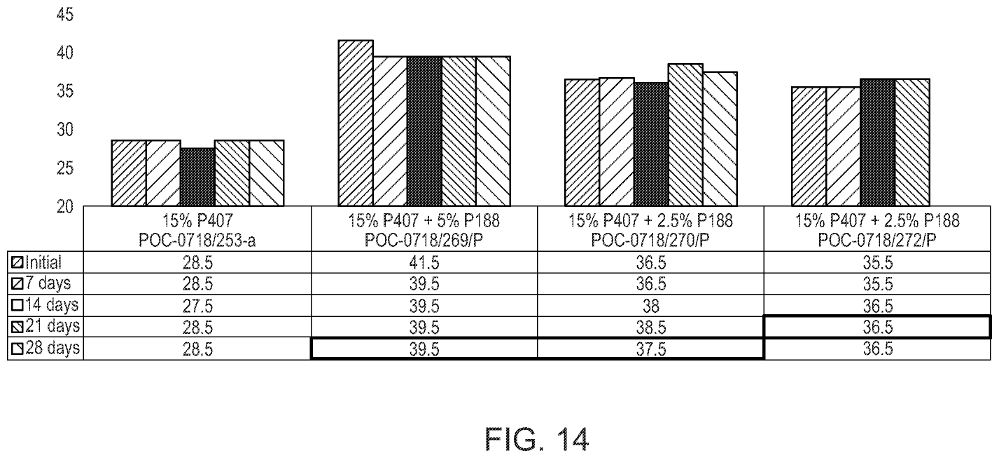

Excipients were studied for compatibility. Constant—15% poloxamer 407 was used. The compatibility study was carried out with the following excipients: Mesalamine—6.67%; Transcutol—10%; EDTA—0.1%; Polocarbophil—0.25%+Tris—0.15%; TPGS—0.2%; NaCl—0.5%; Sodium metabisulfite—0.2%; PVA 30 CPS—1%; Kollidon 30-1%. The following lipids were investigated: DSPC—0.5%+DPCC—0.5%; Phospholipon 90G—1%; Lipoid S 100-1%; Phospholipon 90H—1%; Lecithin—1%. The following combination of poloxamer grades was also studied Poloxamer 188-1.5, 2.5 and 5%. Gelation temperatures for the various excipient combinations are shown in FIG. 12. No or marginal impact (1-2° C. from initial) was found for the following excipients: Transcutol, EDTA, TPGS, NaCl, and PVA. Moderate impact (2-5° C. from initial) was found with Mesalamine. Significant impact to gelation temperature was found with Polycarbophil+TRIS, sodium metabisulfite, xanthan gum, and Kollidon. Sodium metabisulfite, which is a preservative agent used in Rowasa® (Reference listed drug) to prevent the oxidation of mesalamine (prone to degradation by oxidation), has a significant impact on gelation temperature. FIG. 13 shows gelation temperatures for various lipid combinations. No or marginal impact (1-2° C. from initial) was found in combinations with DSPC+DPCC, Phospholipon 90 G, and Lipoid S 100. Moderate impact (2-5° C. from initial) was found with Lecithin and significant impact was exhibited by Phospholipon 90 H. FIG. 14 shows gelation temperatures for Poloxamer 188. All poloxamer combinations were stable for 28 days.

In certain embodiments, a composition with lipoid S 100 may be used. The composition consists of 2 parts: Part 1-Lipid emulsified in the poloxamer dissolved in water (Vehicle) and Part 2-API. An exemplary formulation with lipoid S 100 is shown below in Table 69:

Formulation with Lipoid S 100

| | | POC-0718/291P/ |
|---|---|---|
| Ingredients | POC-0718/278P | POC-0718/314P |
| Poloxamer 407 | 12 | 12 |
| Lipoid s 100 | 2.0 | 2.0 |
| Transcutol | 10 | 10 |
| Purified water | QS to 93.33 | QS to 93.33 |
| Tg (° C.) | 31.5 | 33.5 |
| pH | 7.31 | 7.24 |

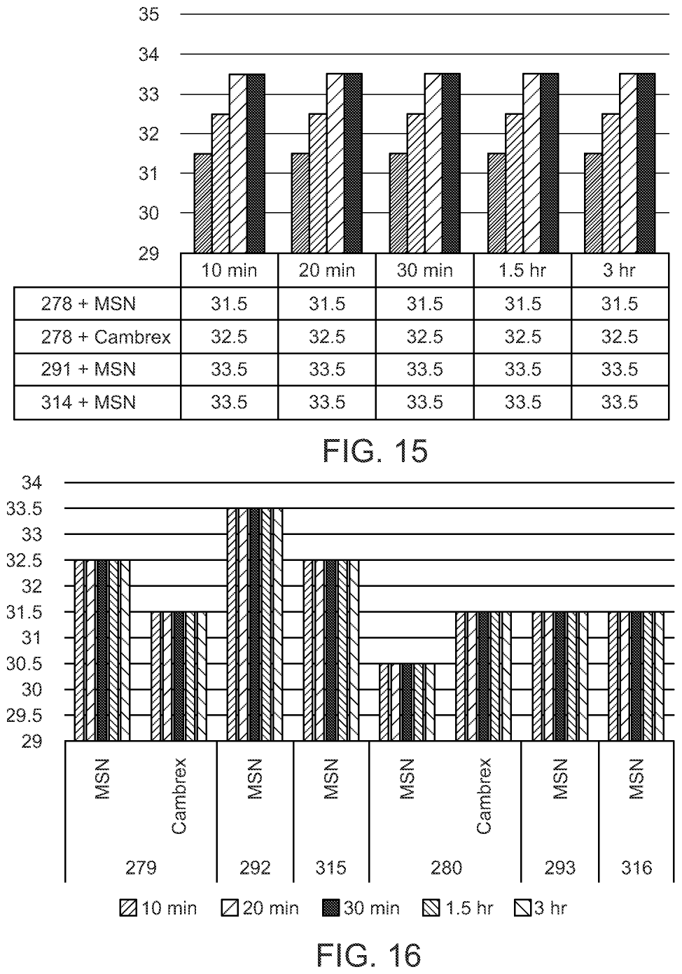

Batch size was 278/314-4 kg and 291-2 kg. A higher gelation temperature was observed with the new batch at the initial time point as seen in FIG. 15 showing gelation temperatures for lipoid S 100 compositions. Tg was stable until 3 hours. MSN and Cambrex (as indicated in FIG. 15) are different mesalamine manufacturers and indicates the source of the test material.

In certain embodiments, a composition with phospholipon 90 G may be used. The composition consists of 2 parts: Part 1-Lipid emulsified in the poloxamer dissolved in water (Vehicle) and Part 2-API. An exemplary formulation with lipoid S 100 is shown below in Table 69:

| | | #292/P/ | | #293/P/ |
|---|---|---|---|---|
| Ingredients | #279/P | #315/P | #280/P | #316/P |
| Edetate sodium | 0.1 | 0.1 | 0.1 | 0.1 |
| Phospholipon | 0.5 | 0.5 | 0.75 | 0.75 |
| 90G | | | | |
| Poloxamer 407 | 12.5 | 12.5 | 12.5 | 12.5 |
| Transcutol P | 10.0 | 10.0 | 10.0 | 10.0 |
| Purified water | QS to 93.33 | QS to 93.33 | QS to 93.33 | QS to 93.33 |

Tg (° C.)

| | | | | |
|---|---|---|---|---|
| Initial | 30.5 | 34.5 | 30.5 | 31.5 |
| pH | 6.12 | | 6.03 | |

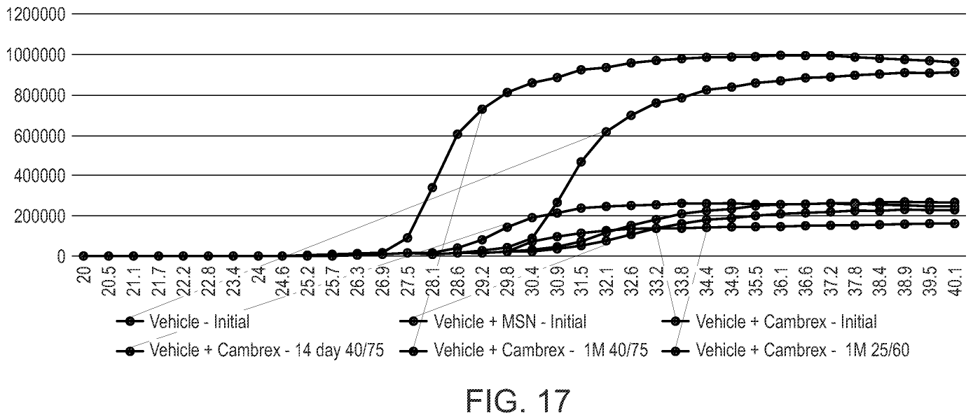

Batch size was 278/315-4 kg and 280/316-4 kg. A higher gelation temperature was observed with the reproducable batches of #279 and #280 as seen in FIG. 16 showing gelation temperatures for phospholipon 90 G compositions. Tg was stable until 3 hours.

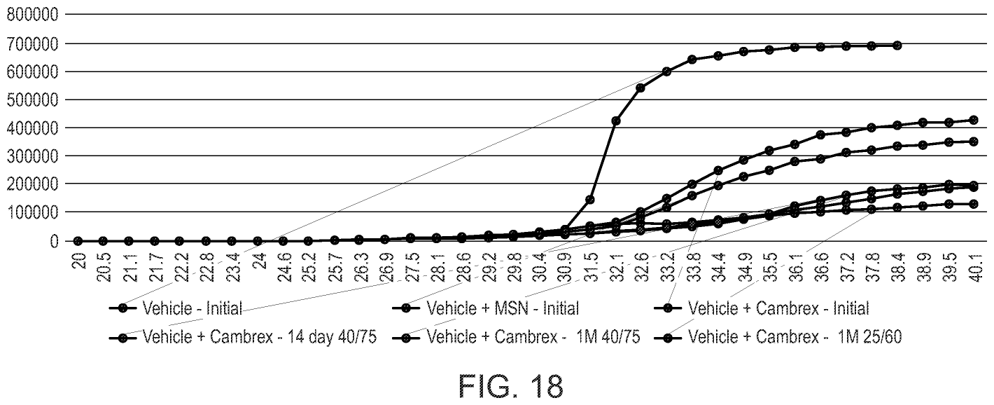

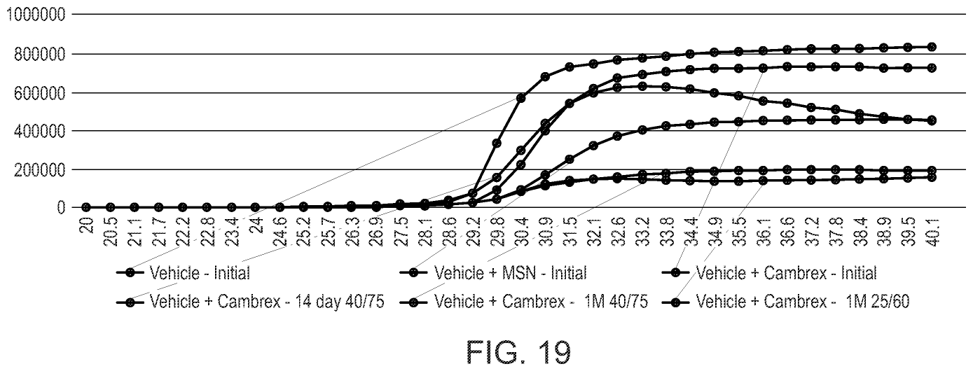

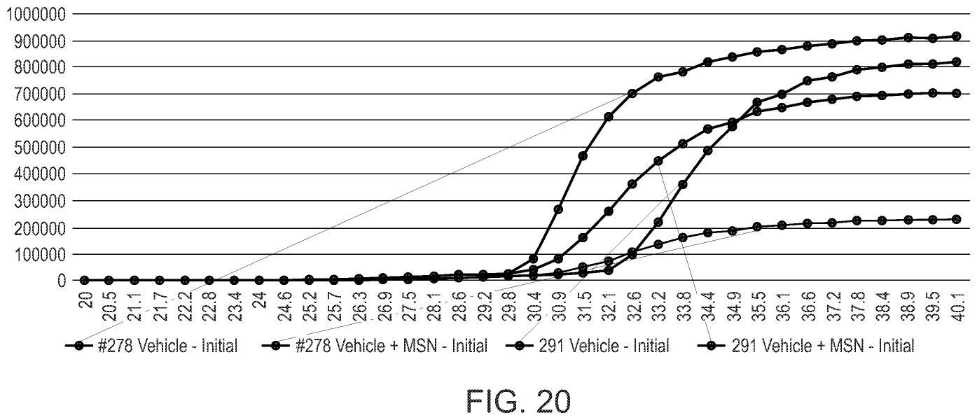

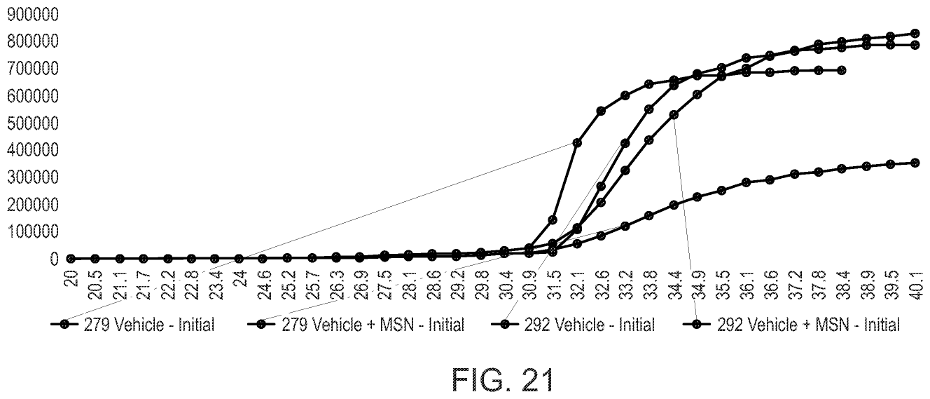

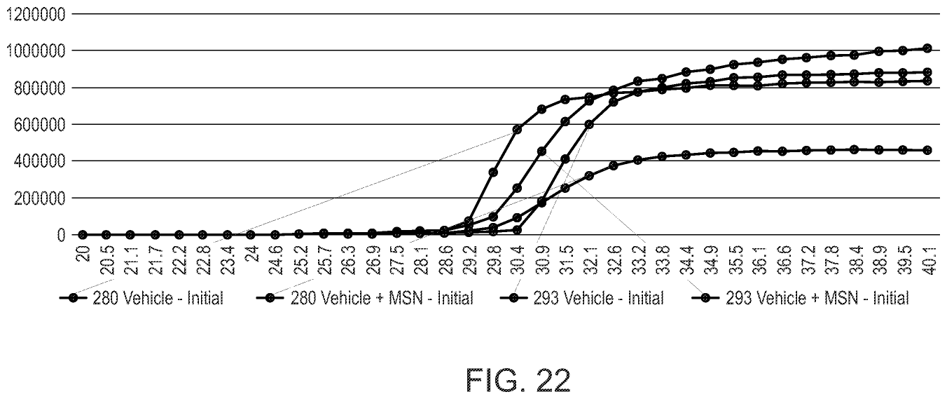

Rheology results are shown for various compositions in FIGS. 17-22. FIG. 17 shows rheology results for composition 278. The reconstituted formulation showed a lower gel strength than the vehicle. The IM 40/75 sample showed lower tg and higher gel strength. FIG. 18 shows rheology results for composition 279. The reconstituted formulation showed a lower gel strength than the vehicle. FIG. 19 shows rheology results for composition 280. The reconstituted formulation showed a lower gel strength than the vehicle (except with cambrex —initial). FIG. 20 shows rheology results for composition 278 vs. composition 291. The 291 composition showed a higher Tg with no significant changes with the addition of API. FIG. 21 shows rheology results for composition 279 vs. composition 292. The 292 composition showed a higher Tg with no significant changes with the addition of API. FIG. 22 shows rheology results for composition 280 vs. composition 293. The 293 composition showed a higher Tg with no significant changes with the addition of API.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A composition comprising:
an active ingredient;
at least one grade of thermogelling polymer, wherein, if present, different grades are present in different concentrations;
a lipid; and
a solubilizer of the lipid.

2. The composition of claim 1, wherein the active ingredient is mesalamine.

3. The composition of claim 2, wherein the concentration of the active ingredient is between about 0.5-15% w/v of the composition.

4. The composition of claim 2, wherein the concentration of the active ingredient is between about 6-8% w/v of the composition.

5. The composition of claim 1, wherein one grade of thermogelling polymer is poloxamer 407.

6. The composition of claim 5, wherein the concentration of poloxamer 407 is between about 10-16% w/v of the composition.

7. The composition of claim 5, wherein the concentration of poloxamer 407 is between about 12-13.5% w/v of the composition.

8. The composition of claim 1, wherein one grade of thermogelling polymer is poloxamer 188.

9. The composition of claim 8, wherein the concentration of poloxamer 188 is between about 0.001-1% w/v of the composition.

10. The composition of claim 1, wherein the lipid is a phospholipid.

11. The composition of claim 10, wherein the phospholipid is phosphatidylcholine.

12. The composition of claim 10, wherein the phosphatidylcholine is a phosphatidylcholine from soybean with agglomerates.

13. The composition of claim 10, wherein the concentration of the lipid is between about 0.001-4% w/v of the composition.

14. The composition of claim 10, wherein the concentration of the lipid is 1.5-2.5% w/v of the composition.

15. The composition of claim 1, wherein the solubilizer of the lipid is diethylene glycol monoethyl ether.

16. The composition of claim 15, wherein the concentration of the solubilizer of the lipid is between about 5-15% w/v of the composition.

17. The composition of claim 15, wherein the concentration of the solubilizer of the lipid is between about 8-10% w/v of the composition.

18. The composition of claim 1, wherein the composition is a liquid at 20-25° C. and transitions to a gel at 29-37° C.

19. A kit comprising:
a first container comprising a first composition comprising an active ingredient; and
a second container comprising a second composition comprising at least one grade of thermogelling polymer wherein, if present, each grade is present in a different concentration, a lipid, and a solubilizer of the lipid.

20. The kit of claim 19, wherein the active ingredient is mesalamine.

21. The kit of claim 20, wherein the concentration of the active ingredient is between about 0.5-15% w/v of the composition.

22. The kit of claim 20, wherein the concentration of the active ingredient is between about 6-8% w/v of the composition.

23. The kit of claim 19, wherein one grade of thermogelling polymer is poloxamer 407.

24. The kit of claim 23, wherein the concentration of poloxamer 407 is between about 10-16% w/v of the composition.

25. The kit of claim 23, wherein the concentration of poloxamer 407 is between about 12-13.5% w/v of the composition.

26. The kit of claim 19, wherein one grade of thermogelling polymer is poloxamer 188.

27. The kit of claim 26, wherein the concentration of poloxamer 188 is between about 0.001-1% w/v of the composition.

28. The kit of claim 19, wherein the lipid is a phospholipid.

29. The kit of claim 28, wherein the phospholipid is phosphatidylcholine.

30. The kit of claim 28, wherein the phosphatidylcholine is a phosphatidylcholine from soybean with agglomerates.

31. The kit of claim 28, wherein the concentration of the lipid is between about 0.001-4% w/v of the composition.

32. The kit of claim 28, wherein the concentration of the lipid is 1.5-2.5% w/v of the composition.

33. The kit of claim 19, wherein the solubilizer of the lipid is diethylene glycol monoethyl ether.

34. The kit of claim 33, wherein the concentration of the solubilizer of the lipid is between about 5-15% w/v of the composition.

35. The kit of claim 33, wherein the concentration of the solubilizer of the lipid is between about 8-10% w/v of the composition.

36. The kit of claim 19, wherein the composition is a liquid at 20-25° C. and transitions to a gel at 29-37° C.

37. A method of treating a condition in a subject, the method comprising the steps of:
receiving a kit comprising a first container comprising a first composition comprising an active ingredient; and a second container comprising a second composition comprising at least one grade of thermogelling polymer wherein, if present, each grade is present in a different concentration, a lipid, and a solubilizer of the lipid.
mixing the first and second compositions to form a final formulation; and
administering the final formulation.

38. The method of claim 37, wherein the condition is an irritable bowel disorder.

39. The method of claim 37, wherein the condition is ulcerative colitis.

40. The method of claim 37, wherein the step of administering is a topical administration.

41. The method of claim 37, wherein the step of administering is an administration of the formulation as an enema.

42. The method of claim 37, wherein the subject performs the steps of mixing and administering.

43. The method of claim 37, wherein the step of mixing comprises adding the second composition to the first composition and shaking for at least 30 seconds to suspend the formulation.

44. The method of claim 37, wherein the step of mixing comprises adding the second composition to the first composition and shaking for at least 15 seconds to suspend the formulation.

45. The method of claim 37, wherein the step of mixing comprises adding the second composition to the first composition and shaking for at least 10 seconds, holding at rest for 1 minute, then shaking again for 10 more seconds to suspend the formulation.

46. The method of claim 37, wherein step of mixing comprises adding the first composition to the second composition and shaking for at least 10 seconds, holding at rest for 1 minute, then shaking again for 10 more seconds to suspend the formulation.

47. The method of claim 37, wherein the active ingredient is mesalamine.

48. The method of claim 47, wherein the concentration of the active ingredient is between about 0.5-15% w/v of the composition.

49. The method of claim 47, wherein the concentration of the active ingredient is between about 6-8% w/v of the composition.

50. The method of claim 37, wherein one grade of thermogelling polymer is poloxamer 407.

51. The method of claim 50, wherein the concentration of poloxamer 407 is between about 10-16% w/v of the composition.

52. The method of claim 50, wherein the concentration of poloxamer 407 is between about 12-13.5% w/v of the composition.

53. The method of claim 37, wherein one grade of thermogelling polymer is poloxamer 188.

54. The method of claim 53, wherein the concentration of poloxamer 188 is between about 0.001-1% w/v of the composition.

55. The method of claim 37, wherein the lipid is a phospholipid.

56. The method of claim 55, wherein the phospholipid is phosphatidylcholine.

57. The method of claim 55, wherein the phosphatidylcholine is a phosphatidylcholine from soybean with agglomerates.

58. The method of claim 55, wherein the concentration of the lipid is between about 0.001-4% w/v of the composition.

59. The method of claim 55, wherein the concentration of the lipid is 1.5-2.5% w/v of the composition.

60. The method of claim 37, wherein the solubilizer of the lipid is diethylene glycol monoethyl ether.

61. The method of claim 60, wherein the concentration of the solubilizer of the lipid is between about 5-15% w/v of the composition.

62. The method of claim 60, wherein the concentration of the solubilizer of the lipid is between about 8-10% w/v of the composition.

63. The method of claim 37, wherein the composition is a liquid at 20-25° C. and transitions to a gel at 29-37° C.
